(19) United States
(12) Patent Application Publication
(10) Pub. No.: US 2009/0035317 A1
(43) Pub. Date: Feb. 5, 2009

(54) Peptides binding to vascular endothelial growth factor

(76) Inventors: Patrick S. Daugherty, Santa Barbara, CA (US); Sophia Kenrick, Santa Barbara, CA (US)

Correspondence Address: ROBINS & PASTERNAK, 1731 EMBARCADERO ROAD, SUITE 230, PALO ALTO, CA, 94303, US

(21) Appl. No.: 12/008,828

(22) Filed: Jan. 14, 2008

Related U.S. Application Data
(60) Provisional application No. 60/880,359, filed on Jan. 12, 2007.

(51) Int. Cl. C07K 14/475; C07K 14/00; C07K 7/06; A61K 39/395; C40B 30/04; A61P 35/00; G01N 33/53; A61K 38/02; C07K 7/08

(52) U.S. Cl. 424/158.1; 530/326; 530/327; 530/324; 530/399; 506/9; 436/501; 514/2

(57) ABSTRACT

The invention relates to peptides that bind to vascular endothelial growth factor (VEGF). The invention further relates to methods of screening for VEGF-binding peptides, methods of using VEGF-binding peptides to detect the presence of VEGF in a biological sample, and methods of using VEGF-binding peptides to modulate VEGF activity and angiogenesis, and as components in a therapeutic or prophylactic composition.

### CROSS-REFERENCE TO RELATED APPLICATION

This application claims benefit under 35 U.S.C. §119(e) of provisional application 60/880,359, filed Jan. 12, 2007, which application is hereby incorporated by reference in its entirety.

### STATEMENT REGARDING FEDERALLY SPONSORED RESEARCH OR DEVELOPMENT

This invention was made with support under NIH Grant U54-CA119335-02-Project 3, from the National Institutes of Health. Accordingly, the United States Government may have certain rights in this invention.

### TECHNICAL FIELD

The disclosure relates to peptides that bind to vascular endothelial growth factor (VEGF). The disclosure further relates to methods of screening for VEGF-binding peptides, methods of using VEGF-binding peptides to detect the presence of VEGF in a biological sample, and methods of using VEGF-binding peptides as components in a therapeutic or prophylactic composition.

### BACKGROUND

The development of the vascular system (sometimes referred to as the vascular tree) involves two major processes: vasculogenesis and angiogenesis. Vasculogenesis is the process by which the major embryonic blood vessels originally develop from early differentiating endothelial cells such as angioblasts and hematopoietic precursor cells that in turn arise from the mesoderm. Angiogenesis is the term used to refer to the formation of the rest of the vascular system that results from vascular sprouting from the pre-existing vessels formed during vasculogenesis (see, e.g., Risau et al. (1988) Devel. Biol., 125:441 450). Both processes are important in a variety of cellular growth processes including developmental growth, tissue regeneration and tumor growth, as all these processes require blood flow for the delivery of necessary nutrients.

Thus, angiogenesis plays a critical role in a wide variety of fundamental physiological processes in the normal individual including embryogenesis, somatic growth, and differentiation of the nervous system. In the female reproductive system, angiogenesis occurs in the follicle during its development, in the corpus luteum following ovulation and in the placenta to establish and maintain pregnancy. Angiogenesis additionally occurs as part of the body's repair processes, such as in the healing of wounds and fractures. Thus, promotion of angiogenesis can be useful in situations in which establishment or extension of vascularization is desirable. Angiogenesis, however, is also a critical factor in a number of pathological processes, perhaps must notably tumor growth and metastasis, as tumors require continuous stimulation of new capillary blood vessels in order to grow. Other pathological processes affected by angiogenesis include conditions associated with blood vessel proliferation, especially in the capillaries, such as diabetic retinopathy, arthropathies, psoriasis and rheumatoid arthritis.

Given its key role in both normal physiological and pathological processes, not surprisingly considerable research effort has been directed towards identifying factors involved in the stimulation and regulation of angiogenesis. A number of growth factors have been purified and characterized. Such factors include fibroblast growth factors (FGFs), platelet-derived growth factor (PDGF), transforming growth factor alpha (TGFα), and hepatocyte growth factor (HGF) (for reviews of angiogenesis regulators, see, e.g., Klagsbrun et al. (1991) Ann. Rev. Physiol., 53:217 39; and Folkman et al. (1992) J. Biol. Chem., 267:10931 934).

Current research indicates that a family of endothelial cell-specific growth factors, the vascular endothelial growth factors (VEGFs), together with their cognate receptors, are primarily responsible for stimulation of endothelial cell growth and differentiation. These factors are members of the PDGF family and appear to act primarily by binding to endothelial receptor tyrosine kinases (RTKs). Five endothelial cell-specific receptor tyrosine kinases have been identified thus far, namely VEGFR-1 (also called Flt-1), VEGFR-2 (also called KDR/Flk-1), VEGFR-3 (Flt4), Tie and Tek/Tie-2, each of which play specific roles in vasculogenesis and angiogenesis.

VEGF dysregulation is associated with numerous pathologies, including cancer, rheumatoid arthritis, diabetic retinopathy and psoriasis. The identification of agents that can modulate or block VEGF signalling is therefore clinically attractive. Inhibitors of VEGF signalling could be used, for example, to suppress tumor growth that is dependent on vascularization of adjacent tissue or neovascularization associated with other disease processes. Thus, there remains a need for compositions and methods for modulating VEGF activity and angiogenesis.

### SUMMARY

The present disclosure relates, in part, to peptides that bind to VEGF. These peptides can be used in a wide range of applications, including as tools to detect the presence of VEGF in a biological sample, as components of a therapeutic or prophylactic composition, and in methods of modulating VEGF activity and angiogenesis in a subject.

In one aspect, provided herein is a peptide comprising a W-E/D-W-E/D motif that binds to VEGF. In certain embodiments the peptide comprises a X₂-G-W-W-L-W-D-W-E-X-G-X₅-R consensus sequence (SEQ ID NO:28), where X₂ is any 2 amino acid residues, and X₅ is any 5 amino acid residues, which may be the same or different. In other embodiments the peptide comprises a A-W-W-L-N-T-W-D-W-E-R-S-N consensus sequence (SEQ ID NO:29). In yet other embodiments the peptide comprises a C-X-I-Q-I-M-W-D-W-E-C-F-R consensus sequence (SEQ ID NO:30). In certain embodiments, the peptide comprises an amino acid sequence selected from the group consisting of SEQ ID NOS: 1-27. In one embodiment, the peptide comprises the amino acid sequence of SEQ ID NO: 18. In another embodiment, the peptide comprises the amino acid sequence of SEQ ID NO:24. In certain embodiments, the peptide comprises a detectable label.

In certain embodiments, the peptide is a bidentate peptide comprising a first VEGF-binding peptide sequence and a second VEGF-binding peptide sequence. The first and second VEGF-binding peptide sequences can be joined by a linker, typically a peptide linker of two or more amino acid residues. Optionally, the peptide sequences can be joined by a non-peptide linker. Such linkers are known to those of skill in the art. In one embodiment, the linker comprises the amino acid sequence of GGGSGGG (SEQ ID NO:52). In certain embodiments, the bidentate VEGF-binding peptide comprises a first peptide sequence selected from the group consisting of SEQ ID NOS:1-31 and a second peptide sequence selected from the group consisting of SEQ ID NOS:42-51. In certain embodiments, the bidentate peptide comprises a sequence selected from the group consisting of SEQ ID NOS:32-41.

In certain embodiments, the peptides bind to VEGF with dissociation constants (K_{D}'s) ranging from 10⁻⁶ M to 10⁻¹² M. For example, a peptide may bind to VEGF with a K_{D} of less than 10⁻⁶ M, a K_{D} of less than 10⁻⁸ M, a K_{D} of less than 10⁻⁹ M, a K_{D} of less than 10⁻¹⁰ M, a K_{D} of less than 10⁻¹¹ M, or a K_{D} of less than 10⁻¹² M.

In another aspect, provided herein is a composition comprising at least one peptide described herein. In certain embodiments, the composition comprises one or more peptides comprising an amino acid sequence selected from the group consisting of SEQ ID NOS:1-30 and 32-51. In one embodiment, the composition comprises a peptide comprising the amino acid sequence of SEQ ID NO: 18. In another embodiment, the composition comprises a peptide comprising the amino acid sequence of SEQ ID NO:24.

In certain embodiments, the composition further comprises a pharmaceutically acceptable excipient and, optionally, one or more therapeutic agents, such as a vascular endothelial growth factor (VEGF)/angiogenesis inhibitor, an anti-cancer agent, or an anti-sepsis agent. VEGF/angiogenesis inhibitors include, but are not limited to, an anti-VEGF/VEGFR antibody, a VEGF receptor tyrosine kinase inhibitor, a VEGF/VEGFR binding aptamer, an antisense inhibitor of VEGF/VEGFR expression, an anti-VEGF/VEGFR ribozyme, an anti-VEGF/VEGFR siRNA, or an endogenous VEGF/angiogenesis inhibitor. Anti-cancer agents include, but are not limited to, agents used in radiation therapy, chemotherapy, immunotherapy, gene therapy, or anti-angiogenic therapy. Anti-sepsis agents include, but are not limited to, antibacterials, antibodies, chemokines and chemokine fragments, C5a peptides, protein C, BPI protein, COX-2 inhibitors, and algae lipopolysaccharides.

In another aspect, provided herein is a complex comprising any of the peptides described herein and VEGF.

In another aspect, provided herein is a method of identifying a peptide comprising a X₆-W-E/D-W-E/D-X₉ motif that binds to VEGF (where X₆ is any 6 amino acid residues, and X₉ is any 9 amino acid residues), the method comprising:
a) providing a library of peptides wherein each peptide in the library comprises the X₆-W-E/D-W-E/D-X₉ motif, and
b) screening the library for peptides that bind to VEGF

In certain embodiments, the peptide library is screened using a virus or cell surface display method, for example, phage display or bacterial display. In one embodiment, the library is screened for peptides that bind to VEGF by magnetic-activated cell sorting (MACS). In certain embodiments, the method further comprises screening the library for peptides that bind to VEGF by fluorescence-activated cell sorting (FACS).

In another aspect, provided herein is a method for modulating angiogenesis in a subject comprising administering an effective amount of a composition comprising at least one peptide comprising an W-E/D-W-E/D motif that binds to VEGF. In certain embodiments at least one peptide comprises a X₂-G-W-W-L-W-D-W-E-X-G-X₅-R consensus sequence (SEQ ID NO:28). In other embodiments at least one peptide comprises a A-W-W-L-N-T-W-D-W-E-R-S-N consensus sequence (SEQ ID NO:29). In yet other embodiments at least one peptide comprises a C-X-I-Q-I-M-W-D-W-E-C-F-R consensus sequence (SEQ ID NO:30). In certain embodiments, the composition comprises one or more peptides comprising an amino acid sequence selected from the group consisting of SEQ ID NOS:1-30 and 32-51. In one embodiment, at least one peptide comprises the amino acid sequence of SEQ ID NO: 18. In another embodiment, at least one peptide comprises the amino acid sequence of SEQ ID NO:24. In certain embodiments, the composition comprises at least one bidentate peptide comprising a first VEGF-binding peptide sequence and a second VEGF-binding peptide sequence. In certain embodiments, the bidentate VEGF-binding peptide comprises a first peptide sequence selected from the group consisting of SEQ ID NOS: 1-31 and a second peptide sequence selected from the group consisting of SEQ ID NOS:42-51. In certain embodiments, the bidentate peptide comprises a sequence selected from the group consisting of SEQ ID NOS:32-41. In one embodiment, the composition inhibits angiogenesis in the subject. In certain embodiments, the composition is administered according to a daily dosing regimen. In certain embodiments, the composition is administered intermittently.

In certain embodiments, the method further comprises administering to a subject a therapeutically effective amount of a vascular endothelial growth factor (VEGF)/angiogenesis inhibitor, for example, an anti-VEGF/VEGF receptor (VEGFR) antibody, a VEGF receptor tyrosine kinase inhibitor, a VEGF/VEGFR binding aptamer, an antisense inhibitor of VEGF/VEGFR expression, an anti-VEGF/VEGFR ribozyme, an anti-VEGF/VEGFR siRNA, or an endogenous VEGF/angiogenesis inhibitor. In certain embodiments, the VEGF/angiogenesis inhibitor is administered according to a daily dosing regimen. In certain embodiments, the VEGF/angiogenesis inhibitor is administered intermittently.

In another aspect, provided herein is a method for inhibiting VEGF activity in a subject comprising administering an effective amount of a composition comprising at least one peptide comprising an W-E/D-W-E/D motif that binds to VEGF. In certain embodiments at least one peptide comprises a X₂-G-W-W-L-W-D-W-E-X-G-X₅-R consensus sequence (SEQ ID NO:28). In other embodiments at least one peptide comprises a A-W-W-L-N-T-W-D-W-E-R-S-N consensus sequence (SEQ ID NO:29). In yet other embodiments at least one peptide comprises a C-X-I-Q-I-M-W-D-W-E-C-F-R consensus sequence (SEQ ID NO:30). In certain embodiments, the composition comprises one or more peptides comprising an amino acid sequence selected from the group consisting of SEQ ID NOS:1-30 and 32-51. In one embodiment, at least one peptide comprises the amino acid sequence of SEQ ID NO: 18. In another embodiment, at least one peptide comprises the amino acid sequence of SEQ ID NO:24. In certain embodiments, the composition comprises at least one bidentate peptide comprising a first VEGF-binding peptide sequence and a second VEGF-binding peptide sequence. In certain embodiments, the bidentate VEGF-binding peptide comprises a first peptide sequence selected from the group consisting of SEQ ID NOS: 1-31 and a second peptide sequence selected from the group consisting of SEQ ID NOS:42-51, for example, the composition may include one or more bidentate peptides comprising a sequence selected from the group consisting of SEQ ID NOS:32-41. In certain embodiments, the composition is administered according to a daily dosing regimen. In certain embodiments, the composition is administered intermittently.

In certain embodiments, the subject has an angiogenesis disorder, cancer, or sepsis. In one embodiment, the method further comprises administering to a subject a therapeutically effective amount of an anti-cancer agent, for example, any agent used in radiation therapy, chemotherapy, immunotherapy, gene therapy, or anti-angiogenic therapy. In another embodiment, the method further comprises administering to a subject a therapeutically effective amount of an anti-sepsis agent, for example, an antibacterial, an antibody, a chemokine or chemokine fragment, a C5a peptide, protein C, BPI protein, a COX-2 inhibitor, or an algae lipopolysaccharide.

In another aspect, provided herein is a method for treating a disorder selected from the group consisting of an angiogenesis disorder, cancer, and sepsis, the method comprising administering to a subject a therapeutically effective amount of a composition comprising at least one peptide comprising an W-E/D-W-E/D motif that binds to VEGF. In certain embodiments at least one peptide comprises a X₂-G-W-W-L-W-D-W-E-X-G-X₅-R consensus sequence (SEQ ID NO:28). In other embodiments at least one peptide comprises a A-W-W-L-N-T-W-D-W-E-R-S-N consensus sequence (SEQ ID NO:29). In yet other embodiments at least one peptide comprises a C-X-I-Q-I-M-W-D-W-E-C-F-R consensus sequence (SEQ ID NO:30). In certain embodiments, the composition comprises one or more peptides comprising an amino acid sequence selected from the group consisting of SEQ ID NOS:1-30 and 32-51. In one embodiment, at least one peptide comprises the amino acid sequence of SEQ ID NO: 18. In another embodiment, at least one peptide comprises the amino acid sequence of SEQ ID NO:24. In certain embodiments, the composition comprises at least one bidentate peptide comprising a first VEGF-binding peptide sequence and a second VEGF-binding peptide sequence. In certain embodiments, the bidentate VEGF-binding peptide comprises a first peptide sequence selected from the group consisting of SEQ ID NOS:1-31 and a second peptide sequence selected from the group consisting of SEQ ID NOS:42-51, for example, the composition may include one or more bidentate peptides comprising a sequence selected from the group consisting of SEQ ID NOS:32-41. The composition may be administered therapeutically or prophylactically, for example, to prevent or delay the onset of one or more symptoms of the disorder or to ameliorate symptoms of the disorder (e.g., to inhibit angiogenesis, tumor growth, or infection). In certain embodiments, the composition is administered according to a daily dosing regimen. In certain embodiments, the composition is administered intermittently. In certain embodiments, the method further comprises administering to the subject one or more other therapeutic agents, such as VEGF/angiogenesis inhibitors, anti-cancer agents, anti-sepsis agents, or other medications used to treat a particular condition or disease.

In another aspect, provided herein is a method for detecting the presence of VEGF in a biological sample. In one embodiment, the method comprises exposing the biological sample suspected of containing VEGF to any of the peptides described herein; and detecting the presence or absence of the peptide bound to the VEGF, if any, in the sample. Peptides may include a detectable label to facilitate detection, for example, a radioactive isotope, fluorescer, chemiluminescer, enzyme, enzyme substrate, enzyme cofactor, enzyme inhibitor, chromophore, dye, metal ion, metal sol, ligand (e.g., biotin or haptens) and the like.

In certain embodiments, the biological sample is obtained from a subject having an angiogenesis disorder, cancer, or sepsis. The amount of VEGF in a biological sample from a subject who has an angiogenesis disorder, cancer or sepsis can be compared to the amount of VEGF in a corresponding biological sample from a normal subject. Multiple samples can be collected from the subject at different time points for the purpose of monitoring changes in the levels of VEGF in the subject over time. In one embodiment, a first biological sample is collected from the subject at one time point and a second biological sample is collected from the subject at a second time point, the first and second biological samples are exposed to one or more VEGF-binding peptides described herein in order to detect the presence or absence of VEGF-binding peptides bound to the VEGF, if any, in the first and second samples, and the amount of VEGF in the first and second samples is compared to determine if the amount of VEGF is increasing or decreasing over time.

These and other embodiments of the subject invention will readily occur to those of skill in the art in view of the disclosure herein.

### BRIEF DESCRIPTION OF THE DRAWINGS

FIG. 1 shows a schematic representation of the library screening process for identifying high affinity VEGF-binding peptides. A library consisting of 2×10⁸ members comprising peptides containing the X₆-W-E/D-W-E/D-X₉ motif was enriched by MACS selection followed by FACS screening for VEGF-binding peptides.

FIG. 2 shows enrichment data for multiple rounds of VEGF sorting.

FIG. 3 shows measurements of peptide dissociation from VEGF (k_{off}) for selected clones.

FIG. 4 shows the dissociation rate constants (k_{off}) for five of the clones.

FIG. 5 shows measurements of the fluorescence remaining after serum addition relative to the initial fluorescence for five of the clones.

FIG. 6 shows sample fluorescence histograms for clone L2.F4.04.

FIG. 7 shows initial screen of bidentate libraries.

FIG. 8 shows increase in fraction of library gated positive for initial, focused, and bidentate libraries.

FIG. 9 shows inhibition by the soluble v114 peptide of VEGF binding by the monodendate parent peptide L2.F4.04 and the bidentate peptide L3.F5A.01.

### DETAILED DESCRIPTION OF THE INVENTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a peptide” includes a mixture of two or more such peptides, and the like.

The terms “polypeptide”, “peptide”, and “amino acid sequence” as used herein generally refers to any compound comprising naturally occurring or synthetic amino acid polymers or amino acid-like molecules including but not limited to compounds comprising amino and/or imino molecules. No particular size is implied by use of the term “peptide”, “oligopeptide” or “polypeptide” and these terms are used interchangeably. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring (e.g., synthetic). Thus, synthetic oligopeptides, dimers, multimers (e.g., tandem repeats, multiple antigenic peptide (MAP) forms, linearly-linked peptides), cyclized, branched molecules and the like, are included within the definition. The terms also include molecules comprising one or more peptoids (e.g., N-substituted glycine residues) and other synthetic amino acids or peptides. (See, e.g., U.S. Pat. Nos. 5,831,005; 5,877,278; and 5,977,301; Nguyen et al. (2000) Chem. Biol. 7(7):463-473; and Simon et al. (1992) Proc. Natl. Acad. Sci. USA 89(20):9367-9371 for descriptions of peptoids). Non-limiting lengths of peptides suitable for use in the present invention includes peptides of 3 to 5 residues in length, 6 to 10 residues in length (or any integer therebetween), 11 to 20 residues in length (or any integer therebetween), 21 to 75 residues in length (or any integer therebetween), 75 to 100 (or any integer therebetween), or polypeptides of greater than 100 residues in length. Typically, polypeptides useful in this invention can have a maximum length suitable for the intended application. In certain embodiments, the polypeptide is between about 3 and 100 residues in length. Generally, one skilled in art can easily select the maximum length in view of the teachings herein. Further, peptides as described herein, for example synthetic peptides, may include additional molecules such as labels or other chemical moieties (e.g., amyloid specific dyes such as Control Red or Thioflavin). Such moieties may further enhance interaction of the peptides with VEGF and/or further detection of VEGF polypeptides.

Thus, reference to peptides also includes derivatives of the amino acid sequences of the invention including one or more non-naturally occurring amino acid. A first polypeptide is “derived from” a second polypeptide if it is (i) encoded by a first polynucleotide derived from a second polynucleotide encoding the second polypeptide, or (ii) displays sequence identity to the second polypeptide as described herein. Sequence (or percent) identity can be determined as described below. In certain embodiments, derivatives exhibit at least about 50% percent identity, at least about 80% identity, or even more, such as between about 85% and 99% identity (or any value therebetween) to the sequence from which they were derived. Such derivatives can include postexpression modifications of the polypeptide, for example, glycosylation, acetylation, phosphorylation, and the like.

Amino acid derivatives can also include modifications to the native sequence, such as deletions, additions and substitutions (generally conservative in nature), so long as the polypeptide maintains the desired activity. These modifications may be deliberate, as through site-directed mutagenesis, or may be accidental, such as through mutations of hosts that produce the proteins or errors due to PCR amplification. Furthermore, modifications may be made that have one or more of the following effects: increasing affinity and/or specificity for VEGF proteins and facilitating cell processing (e.g., secretion, peptide presentation, etc.). Polypeptides described herein can be made recombinantly, synthetically, or in tissue culture.

The term “polynucleotide”, as known in the art, generally refers to a nucleic acid molecule. A “polynucleotide” can include both double- and single-stranded sequences and refers to, but is not limited to, prokaryotic sequences, eukaryotic mRNA, cDNA from viral, prokaryotic or eukaryotic mRNA, genomic RNA and DNA sequences from viral (e.g. RNA and DNA viruses and retroviruses), prokaryotic DNA or eukaryotic (e.g., mammalian) DNA, and especially synthetic DNA sequences. The term also captures sequences that include any of the known base analogs of DNA and RNA, and includes modifications such as deletions, additions and substitutions (generally conservative in nature), to the native sequence. These modifications may be deliberate, as through site-directed mutagenesis, or may be accidental, such as through mutations of hosts including polynucleotides encoding VEGF-binding peptides. Modifications of polynucleotides may have any number of effects including, for example, facilitating expression of the polypeptide product in a host cell.

A polynucleotide can encode a biologically active (e.g., VEGF-binding) protein or polypeptide. Depending on the nature of the polypeptide encoded by the polynucleotide, a polynucleotide can include as little as 10 nucleotides, e.g., where the polynucleotide encodes an antigen or epitope. Typically, the polynucleotide encodes peptides of at least 10, 11, 12, 13, 14, 15, 16, 17, 18, 19, 20, 21, 22, 23, 24, 25, 30 or even more amino acids.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein, polypeptide, or peptide means a polypeptide produced by expression of a recombinant polynucleotide. In general, the gene of interest is cloned and then expressed in transformed organisms, as described further below. The host organism expresses the foreign gene to produce the protein under expression conditions.

A “polynucleotide coding sequence” or a sequence that “encodes” a selected polypeptide, is a nucleic acid molecule that is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A transcription termination sequence may be located 3′ to the coding sequence. Typical “control elements,” include, but are not limited to, transcription regulators, such as promoters, transcription enhancer elements, transcription termination signals, and polyadenylation sequences; and translation regulators, such as sequences for optimization of initiation of translation, e.g., Shine-Dalgarno (ribosome binding site) sequences, Kozak sequences (i.e., sequences for the optimization of translation, located, for example, 5′ to the coding sequence), leader sequences (heterologous or native), translation initiation codon (e.g., ATG), and translation termination sequences. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is induced by an analyte, cofactor, regulatory protein, etc.), and constitutive promoters.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

By “isolated” is meant, when referring to a polynucleotide or a polypeptide, that the indicated molecule is separate and discrete from the whole organism with which the molecule is found in nature or, when the polynucleotide or polypeptide is not found in nature, is sufficiently free of other biological macromolecules so that the polynucleotide or polypeptide can be used for its intended purpose.

A peptide is said to “interact” with another peptide or protein if it binds specifically (e.g., in a lock-and-key type mechanism), non-specifically or in some combination of specific and non-specific binding. A first peptide “interacts preferentially” with a second peptide or protein if it binds (non-specifically and/or specifically) to the second peptide or protein with greater affinity and/or greater specificity than it binds to other peptides (e.g., binds to VEGF to a greater degree than to other proteins). The term “affinity” refers to the strength of binding and can be expressed quantitatively as a dissociation constant (K_{d}). It is to be understood that specific binding does not necessarily require interaction between specific amino acid residues and/or motifs of each peptide. For example, in certain embodiments, the peptides described herein interact preferentially with VEGF but, nonetheless, may be capable of binding other polypeptides at a weak, yet detectable, level (e.g., 10% or less of the binding shown to the polypeptide of interest). Typically, weak binding, or background binding, is readily discernible from the preferential interaction with the compound or polypeptide of interest, e.g., by use of appropriate controls.

The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to fluorescein, rhodamine, dansyl, umbelliferone, Texas red, luminol, acradimum esters, NADPH, beta-galactosidase, horseradish peroxidase, glucose oxidase, alkaline phosphatase and urease.

The term “VEGF/angiogenesis inhibitor” as used herein refers to any molecule (e.g., small molecule inhibitor, protein, peptide, nucleic acid, oligonucleotide, antibody, or fragment thereof) that inhibits VEGF/VEGF receptor (VEGFR) activity, and/or VEGF/VEGFR expression, and/or the VEGF signaling pathway, and/or angiogenesis. Inhibition may be complete or partial (i.e., all activity, some activity, or most activity is blocked by an inhibitor). VEGF/angiogenesis inhibitors include, but are not limited to, an anti-VEGF/VEGFR antibody, a VEGF receptor tyrosine kinase inhibitor, a VEGF/VEGFR binding aptamer, an antisense inhibitor of VEGF/VEGFR expression, an anti-VEGF/VEGFR ribozyme, an anti-VEGF/VEGFR siRNA, and an endogenous VEGF/angiogenesis inhibitor.

The term “anti-sepsis agent” as used herein refers to any molecule that can be used to treat or prevent sepsis. Anti-sepsis agents include, but are not limited to, antibacterials, antibodies (e.g., polyclonal immunoglobulins against Gram-negative bacteria, anti-IL-8 antibodies, anti-IL-18 antibodies, anti-C5a antibodies, anti-CD14 antibodies, anti-TNFα antibodies, and anti-bacterial lipopolysaccharide (LPS) antibodies), chemokines and chemokine fragments, C5a peptides, protein C, BPI protein, COX-2 inhibitors, and algae lipopolysaccharides.

The term “anti-cancer agent” as used herein refers to any agent that can be used to treat or prevent cancer, including agents used in radiation therapy, chemotherapy, immunotherapy, gene therapy, or anti-angiogenic therapy. Anti-cancer agents include, but are not limited to, chemotherapy agents, such as topoisomerase I inhibitors (CPT-11 (irinotecan), camptothecin, topotecan); topoisomerase II inhibitors (doxorubicin, daunorubicin); alkylators (temozolomide, carmustine, lomustine, dacarbazine, DTIC, cytoxin, procarbazine); inhibitors of protein kinase C and/or cyclin dependent kinases (flavopiridol, staurosporine, UCN-01, paullones, indirubins, Roscovitine, Purvalanol); inhibitors of farnesyltransferase (ZARNESTRA™ (R115777), Sarazar (SCH66336); inhibitors of histone deacetylase (BMS-214662, Trichostatin A, Trapoxin, MS-27-275, FR901228); inhibitors of HMG-CoA (Mevastatin, Lovastatin); inhibitors of Cdk2,4,6 (retinoids, Fenretinide); EGFR tyrosine kinase inhibitors (Iressa, Gefitinib), Tarceva (Erlotinib); proteasome inhibitor that increases p21/decreases Cdk1 (PS-341); compounds that decrease Cdk1 (arsenic trioxide); platinium compounds (carboplatin, cis-platin, oxaloplatin); anti-angiogenic agents (Avastin, VEGF trap, PTK787, AEE788); antimetabolites (5-fluorouricil, xeloda, methotrexate, Ara-C, depo-Ara-C, 6-thioguanine); Vinca alkaloids (vincristine, vinblastine); taxanes (Taxol, Taxotere), PI3K/Akt/mTor inhibitors, and/or RAD001. Typically, chemotherapy includes an alkylating agent, mitotic inhibitor, antibiotic, or antimetabolite. Chemotherapy may include temozolomide, epothilones, melphalan, carmustine, busulfan, lomustine, cyclophosphamide, dacarbazine, polifeprosan, ifosfamide, chlorambucil, mechlorethamine, busulfan, cyclophosphamide, carboplatin, cisplatin, thiotepa, capecitabine, streptozocin, bicalutamide, flutamide, nilutamide, leuprolide acetate, doxorubicin hydrochloride, bleomycin sulfate, daunorubicin hydrochloride, dactinomycin, liposomal daunorubicin citrate, liposomal doxorubicin hydrochloride, epirubicin hydrochloride, idarubicin hydrochloride, mitomycin, doxorubicin, valrubicin, anastrozole, toremifene citrate, cytarabine, fluorouracil, fludarabine, floxuridine, interferon α-2a, interferon α-2b, interleukin-2, plicamycin, mercaptopurine, methotrexate, medroxyprogersterone acetate, estramustine phosphate sodium, estradiol, leuprolide acetate, megestrol acetate, octreotide acetate, deithylstilbestrol diphosphate, testolactone, goserelin acetate, etoposide phosphate, vincristine sulfate, etoposide, vinblastine, etoposide, vincristine sulfate, teniposide, trastuzumab, gemtuzumab ozogamicin, rituximab, exemestane, irinotecan hydrocholride, asparaginase, gemcitabine hydrochloride, altretamine, topotecan hydrochloride, hydroxyurea, cladribine, mitotane, procarbazine hydrochloride, vinorelbine tartrate, pentrostatin sodium, mitoxantrone, pegaspargase, denileukin difitix, altretinoin, porfimer, bexarotene, paclitaxel, docetaxel, arsenic trioxide, tretinoin, or any combination thereof.

The term “derived from” is used herein to identify the original source of a molecule but is not meant to limit the method by which the molecule is made which can be, for example, by chemical synthesis or recombinant means.

The terms “variant,” “analog” and “mutein” refer to biologically active derivatives of the reference molecule that retain desired activity, such as VEGF-binding activity as described herein. In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and 5,977,301; Nguyen et al., Chem. Biol. (2000) 7:463-473; and Simon et al., Proc. Natl. Acad. Sci. USA (1992) 89:9367-9371 for descriptions of peptoids). In one embodiment, the analog or mutein has at least the same VEGF-binding activity as the native molecule. Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

By “derivative” is intended any suitable modification of the native polypeptide of interest, of a fragment of the native polypeptide, or of their respective analogs, such as glycosylation, phosphorylation, polymer conjugation (such as with polyethylene glycol), or other addition of foreign moieties, so long as the desired biological activity of the native polypeptide is retained. Methods for making polypeptide fragments, analogs, and derivatives are generally available in the art.

By “fragment” is intended a molecule consisting of only a part of the intact full-length sequence and structure. The fragment can include a C-terminal deletion an N-terminal deletion, and/or an internal deletion of the peptide. Active fragments of a particular protein or peptide will generally include at least about 5-10 contiguous amino acid residues of the full-length molecule, in some embodiments, at least about 15-25 contiguous amino acid residues of the full-length molecule, and in other embodiments, at least about 20-50 or more contiguous amino acid residues of the full-length molecule, or any integer between 5 amino acids and the full-length sequence, provided that the fragment in question retains biological activity, such as VEGF-binding activity, as defined herein.

“Substantially purified” generally refers to isolation of a substance (compound, polynucleotide, protein, polypeptide, polypeptide composition) such that the substance comprises the majority percent of the sample in which it resides. Typically in a sample, a substantially purified component comprises at least 50%, in some cases at least 80%-85%, or as much as 90-95% or more of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Homology” refers to the percent identity between two polynucleotide or two polypeptide moieties. Two nucleic acid, or two polypeptide sequences are “substantially homologous” to each other when the sequences exhibit at least about 50%, at least about 75%, at least about 80%-85%, at least about 90%, and at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules (the reference sequence and a sequence with unknown % identity to the reference sequence) by aligning the sequences, counting the exact number of matches between the two aligned sequences, dividing by the length of the reference sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The terms “effective amount” or “pharmaceutically effective amount” of a composition, peptide, or agent (e.g., VEGF/angiogenesis inhibitor, anti-cancer agent, or anti-sepsis agent), as provided herein, refer to a nontoxic but sufficient amount of the composition to provide the desired response, such as modulation (e.g., enhancing or inhibiting) of VEGF activity in a subject, and optionally, a corresponding therapeutic effect. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation.

By “therapeutically effective dose or amount” of a VEGF-binding peptide or a VEGF/angiogenesis inhibitor, anti-cancer agent, or anti-sepsis agent is intended an amount that, when the VEGF-binding peptide or VEGF/angiogenesis inhibitor, anti-cancer agent, or anti-sepsis agent are administered separately or in combination as described herein, brings about a positive therapeutic response, such as inhibiting angiogenesis, tumor growth, or infection. The therapeutically effective dose may be administered prophylactically to prevent or delay the onset of symptoms or therapeutically to ameliorate symptoms of the condition or disorder being treated.

“Treatment” or “treating” a disorder or condition, such as an angiogenesis disorder, cancer, or sepsis can include prophylactic and/or therapeutic administration of a VEGF-binding peptide so as to prevent, delay, and/or treat symptoms of angiogenesis, tumor growth, or infection. In some embodiments, treatment encompasses prophylactic administration. In other embodiments, treatment does not encompass prophylactic administration.

The terms “subject”, “individual” or “patient” are used interchangeably herein and refer to a vertebrate. By “vertebrate” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

As used herein, a “biological sample” refers to a sample of tissue or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies. In particular, biological samples may be obtained from a subject suspected of having an angiogenic disorder, cancer, or sepsis.

### II. GENERAL

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of novel VEGF-binding peptides. Such VEGF-binding peptides may be used in a wide range of applications, including as tools to detect the presence of VEGF in a biological sample, as components of a therapeutic or prophylactic composition, and in methods of modulating VEGF activity in a subject, e.g., for treating or preventing angiogenesis, cancer, or sepsis. In order to further an understanding of the invention, a more detailed discussion is provided below regarding the VEGF-binding peptides and their use in diagnostic and therapeutic applications.

### A. Peptides

In one aspect, the invention provides peptides capable of binding, and in some cases, specifically binding to a VEGF polypeptide. As shown in Example 1, peptides with high affinities for VEGF can be identified by screening libraries of peptides containing the VEGF binding motif X₆-W-E/D-W-E/D-X₉, where X₆ is any 6 amino acid residues, and X₉ is any 9 amino acid residues, which may be the same or different. Peptide display methods such as bacteriophage display (see, e.g., Scott and Smith (1990) Science 249:386-904; Norris et al. (1999) Science 285:744-765; Arap et al. (1998) Science 279:377-806; and Whaley et al. (2000) Nature 405:665-668; Smith (1985) Science 228:1315-1317; herein incorporated by reference in their entireties) or cell surface display (see, e.g., Boder and Wittrup (1997) Nat. Biotechnol. 15:553-557; Georgiou et al. (1997) Nat. Biotechnol. 15:29-34; Shusta et al. (1999) Curr. Opin. Biotechnol. 10:117-122; Wittrup (2001) Curr. Opin. Biotechnol. 12:395-399; Lee et al. (2003) Trends Biotechnol. 21:45-52; herein incorporated by reference in their entireties) can be used in the practice of the invention. In one embodiment, bacterial display is used to identify peptides with high affinities for VEGF (see, e.g., Rice et al. (2006) Protein Sci. 15:825-836; U.S. Patent Application Publication No. 2005/0196406; herein incorporated by reference in their entireties). Cell surface display methods can be used in combination with magnetic-activated cell sorting (MACS) and fluorescence-activated cell sorting (FACS) techniques for quantitative library analysis and screening for VEGF-binding peptides (see, e.g., Rice et al. (2006) Protein Sci. 15:825-836; U.S. Patent Application Publication No. 2005/0196406; Daugherty et al. (2000) J. Immunol. Methods 243(1-2):211-2716; Georgiou (2000) Adv. Protein Chem. 55:293-315; Daugherty et al. (2000) Proc. Natl. Acad. Sci. U.S.A. 97(5):2029-3418; Olsen et al. (2003) Methods Mol. Biol. 230:329-342; and Boder et al. (2000) Proc. Natl. Acad. Sci. U.S.A. 97(20):10701-10705; herein incorporated by reference in their entireties). Multiple rounds of selection in the presence of a VEGF polypeptide are usually required to sufficiently enrich libraries for peptides that bind with high affinity to the target VEGF polypeptide.

In certain embodiments, peptides bind to VEGF with dissociation constants (K_{D}'s) ranging from 10⁻⁶ M to 10⁻¹² M. For example, a peptide may bind to VEGF with a K_{D} of less than 10⁻⁶ M, a K_{D} of less than 10⁻⁸ M, a K_{D} of less than 10⁻⁹ M, a K_{D} of less than 10⁻¹⁰ M, a K_{D} of less than 10⁻¹¹ M, or a K_{D} of less than 10⁻¹² M.

The sequences of exemplary VEGF-binding peptides identified using bacterial display are shown in Table 1:

TABLE 1

| | |
|---|---|
| SEQ ID NO | Sequence |

| | |
|---|---|
| 1 | SLGWWLWDWEKQVQLDRRK |
| 2 | AWWLQRWDWEDRQGERGMW |
| 3 | MGNKQGWEWDYFRWVQMAA |
| 4 | HQQLQYWDWIEAVVHNG |
| 5 | AAGWWLWDWEAGRQLRDRL |
| 6 | TACMGGWEWDYWRAMSVGH |
| 7 | VWHQVPWDWEMLLGKNLDR |
| 8 | YSVSRMWDWEKVKWVPWPA |
| 9 | GPGWWLWDWDNLGARRGGL |
| 10 | GAALTRWEWEVYKLVENCM |
| 11 | GWWLNSWDWEHNTSLGPGV |
| 12 | LLGWWLWDWDGARSMNGRW |
| 13 | WMGWSTWEWDRGGMMRSST |
| 14 | AWWLRTWDWERSNH |
| 15 | PGGWWLWDWERVRGGEHLR |
| 16 | DKPWWLWDWEKGQVGSSRS |
| 17 | GESWWLWDWDWGSKRQLVA |
| 18 | CPVQTMWDWECMRAFIEG |
| 19 | GWWLSTWEWERSALAAEQK |
| 20 | WKELRFWDWEGGNHKKCVT |
| 21 | WKELRFWDWEGGNHKKCVT |
| 22 | WRLLPTWDWDWGQSSTETM |
| 23 | WCPLSGWDWEGSVCRSGGS |
| 24 | NFGYGKWEWDYGKWLEKVG |
| 25 | CLRQIIWDWECFRTNNTMV |
| 26 | WIPLQLWDWERDSCSTIGL |
| 27 | YVNLWGWEGWGGQSEQ |

As shown in Example 1, three consensus sequences for VEGF binding were identified by alignment of peptides with high affinities for VEGF: W-L-W-D-W-E-X-G-X₅-R (SEQ ID NO:28), A-W-W-L-N-T-W-D-W-E-R-S-N (SEQ ID NO:29), and C-X-I-Q-I-M-W-D-W-E-C-F-R (SEQ ID NO:30). Peptides comprising the sequences of SEQ ID NOS:1-27 or variants thereof that bind to VEGF, or peptides comprising the consensus sequences of SEQ ID NOS:28-30 are useful in the compositions and methods described herein.

Peptides with increased affinity and specificity for VEGF can be identified by screening libraries for bidentate VEGF-binding peptides (see Example 2). Such bidentate peptides comprise a first VEGF-binding peptide sequence and a second VEGF-binding peptide sequence, which are joined by a linker. The linker is typically a peptide linker of two or more amino acid residues. Optionally, the peptide sequences can be joined by a non-peptide linker. The linker is used to separate the first and second VEGF-binding peptides by a distance sufficient to permit cooperative binding of the first and second VEGF-binding peptides to VEGF. Linkers can be incorporated into the bidentate VEGF-binding peptides using standard techniques well known in the art. The sequences of exemplary bidentate VEGF-binding peptides are shown in Table 2:

TABLE 2

| | |
|---|---|
| SEQ | |
| ID | Sequence |

| | | | |
|---|---|---|---|
| NO | Peptide 1 | Linker | Peptide 2 |

| | | | |
|---|---|---|---|
| 32 | SSKCGRWGETPCLAE | GGGSGGG | NFGYGKWEWDYGKWLEKVG |
| 33 | VTSRYEMREDGSWKK | GGGSGGG | NFGYGKWEWDYGKWLEKVG |
| 34 | AKRSSYSMWGAMP | GGGSGGG | NFGYGKWEWDYGKWLEKV |
| 35 | SYVLKEWSVPSWGKP | GGGSGGG | NFGYGKWEWDYGKWLEKV |
| 36 | SKLKTLLWQQ | GGGSGGG | NFGYGKWEWDYGKWLEKV |
| 37 | LLQKVMAKAGRGLPR | GGGSGGG | CPVQTMWDWECMRAFIEG |
| 38 | GQSLA | GGGSGGG | CPVQTMWDWECMRAFIEG |
| 39 | GQSL | GGGSGGG | CPVQTMWDWECMRAFIEG |
| 40 | ALSKATEGVLRSLRG | GGGSGGG | CPVQTMWDWECMRAFIEG |
| 41 | LLRVLTAAVGSSRGV | GGGSGGG | CPVQTMWDWECMRAFIEG |

Bidentate VEGF-binding peptides comprising a first peptide sequence selected from the group consisting of SEQ ID NOS:1-31 and a second peptide sequence selected from the group consisting of SEQ ID NOS:42-51, and bidentate peptides comprising a sequence selected from the group consisting of SEQ ID NOS:32-41, or variants thereof, are useful in the compositions and methods described herein.

In certain embodiments, the peptides described herein may include one or more substitutions, additions, and/or mutations. For example, one or more residues may be replaced in the peptides with other residues, for example alanine residues or with an amino acid analog or peptoid.

Furthermore, the peptides described herein may also include additional peptide or non-peptide components. Non-limiting examples of additional peptide components include spacer residues, for example two or more glycine (natural or derivatized) residues on one or both ends or residues that may aid in solubilizing the peptides, for example acidic residues such as aspartic acid (Asp or D).

Non-limiting examples of non-peptide components (e.g., chemical moieties) that may be added to the peptides described herein include, one or more labels, tags (e.g., biotin), dyes and the like, at either terminus or internal to the peptide. The non-peptide components may also be attached (e.g., via covalent attachment of one or more labels), directly or through a spacer (e.g., an amide group), to position(s) on the compound that are predicted by quantitative structure-activity data and/or molecular modeling to be non-interfering. Derivatization (e.g., labeling, cyclizing, attachment of chemical moieties, etc.) of compounds should not substantially interfere with (and may even enhance) the binding properties, biological function and/or pharmacological activity of the peptide.

The peptides as described herein interact preferentially with VEGF and, accordingly, are useful in a wide range of detection, diagnostic and therapeutic applications. For example, in embodiments in which the peptide interacts preferentially with VEGF, the peptides themselves can be used to detect aberrant expression (i.e., overexpression or underexpression) of VEGF in a biological sample, such as a tissue or organ sample from a subject who has an angiogenesis disorder, cancer, or sepsis. The peptides described herein are also useful in therapeutic compositions (e.g., to bind to VEGF and modulate VEGF activity and/or angiogenesis).

The interaction of the peptides with VEGF proteins can be tested using any known binding assay, for example standard immunoprecipitation assays such as ELISAs, Western blots and the like. Peptides as described herein can be added adsorbed onto a solid support (as further described below) and used to obtain a quantitative value directly related to the number of peptide-VEGF binding interactions on the solid support. Variations and other assays known in the art can also be used to demonstrate the specificity of the peptides of the invention. (See, also, Example 1).

Thus, non-limiting examples of methods of evaluating binding specificity and/or affinity of the peptides described herein include standard Western and Far-Western Blotting procedures; labeled peptides; ELISA-like assays; and/or cell based assays. Western blots, for example, typically employ a tagged primary antibody that detects denatured VEGF protein from an SDS-PAGE gel that has been electroblotted onto nitrocellulose or PVDF. The primary antibody is then detected (and/or amplified) with a probe for the tag (e.g., streptavidin-conjugated alkaline phosphatase, horseradish peroxidase, ECL reagent, and/or amplifiable oligonucleotides). Binding can also be evaluated using detection reagents such as a peptide with an affinity tag (e.g., biotin) that is labeled and amplified with a probe for the affinity tag (e.g., streptavidin-conjugated alkaline phosphatase, horseradish peroxidase, ECL reagent, or amplifiable oligonucleotides). In addition, microtitre plate procedures similar to sandwich ELISA may be used, for example, a VEGF-specific peptide as described herein is used to immobilize VEGF protein(s) on a solid support (e.g., well of a microtiter plate, bead, etc.) and an additional detection reagent which could include, but is not limited to, another VEGF-specific peptide with an affinity and/or detection label such as a conjugated alkaline phosphatase, horseradish peroxidase, ECL reagent, or amplifiable oligonucleotides. Cell based assays can also be employed, for example, where the VEGF protein is detected directly on individual cells (e.g., using a fluorescently labeled VEGF-specific peptide that enables fluorescence based cell sorting, counting, or detection of the specifically labeled cells).

### B. Polypeptide Production

The peptides of the present invention can be produced in any number of ways, all of which are well known in the art.

In one embodiment, the polypeptides are generated using recombinant techniques, well known in the art. One of skill in the art could readily determining nucleotide sequences that encode the desired peptide using standard methodology and the teachings herein.

Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Sambrook et al., supra, for a description of techniques used to obtain and isolate DNA.

The sequences encoding the peptide can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding polypeptides useful in the claimed peptides that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci. USA (1982) 79:6409.

Once coding sequences have been isolated and/or synthesized, they can be cloned into any suitable vector or replicon for expression. (See, also, Examples). As will be apparent from the teachings herein, a wide variety of vectors encoding modified polypeptides can be generated by creating expression constructs which operably link, in various combinations, polynucleotides encoding polypeptides having deletions or mutations therein.

Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit).

Plant expression systems can also be used to produce the peptides described herein. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

Viral systems, such as a vaccinia based infection/transfection system, as described in Tomei et al., J. Virol. (1993) 67:4017-4026 and Selby et al., J. Gen. Virol. (1993) 74:1103-1113, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. With the present invention, both the naturally occurring signal peptides or heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the proteins of the present invention are produced by growing host cells transformed by an expression vector described above under conditions whereby the protein of interest is expressed. The selection of the appropriate growth conditions is within the skill of the art.

In one embodiment, the transformed cells secrete the polypeptide product into the surrounding media. Certain regulatory sequences can be included in the vector to enhance secretion of the protein product, for example using a tissue plasminogen activator (TPA) leader sequence, an interferon (γ or α) signal sequence or other signal peptide sequences from known secretory proteins. The secreted polypeptide product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the recombinant polypeptides substantially intact. Intracellular proteins can also be obtained by removing components from the cell wall or membrane, e.g., by the use of detergents or organic solvents, such that leakage of the polypeptides occurs. Such methods are known to those of skill in the art and are described in, e.g., Protein Purification Applications: A Practical Approach, (E. L. V. Harris and S. Angal, Eds., 1990).

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced polypeptides are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining the intracellular polypeptides of the present invention involves affinity purification, such as by immunoaffinity chromatography using antibodies (e.g., previously generated antibodies), or by lectin affinity chromatography. In particular, lectin resins that recognize mannose moieties can be used, such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA) and Allium ursinum agglutinin (AUA). The choice of a suitable affinity resin is within the skill in the art. After affinity purification, the polypeptides can be further purified using conventional techniques well known in the art, such as by any of the techniques described above.

Peptides can be conveniently synthesized chemically, for example by any of several techniques that are known to those skilled in the peptide art. In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. These methods are typically used for relatively small polypeptides, i.e., up to about 50-100 amino acids in length, but are also applicable to larger polypeptides.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

The polypeptide analogs of the present invention can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

### C. Pharmaceutical Compositions

The VEGF-binding peptides can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the VEGF-binding peptide or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines, fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the VEGF-binding peptide (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, in some cases, from about 5% to about 98% by weight, in other cases, from about 15 to about 95% by weight of the excipient. In one embodiment, the concentration of the excipient is less than 30% by weight. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional compositions include those for oral, ocular, or localized delivery.

The pharmaceutical preparations herein can also be housed in a syringe, an implantation device, or the like, depending upon the intended mode of delivery and use. In one embodiment, the compositions comprising one or more VEGF-binding peptides described herein are in unit dosage form, meaning an amount of a conjugate or composition of the invention appropriate for a single dose, in a premeasured or pre-packaged form.

The compositions herein may optionally include one or more additional agents, such as VEGF/angiogenesis inhibitors, anti-cancer agents, anti-sepsis agents, or other medications used to treat a subject for a condition or disease. For example, compositions can include one or more VEGF/angiogenesis inhibitors such as anti-VEGF/VEGFR antibodies, for example, Avastin® (bevacizumab; Genentech, South San Francisco Calif.), Lucentis® (ranibizumab; Genentech, South San Francisco Calif.), HuMV833, 2C3, and 2C7; VEGFR tyrosine kinase inhibitors, for example, SU011248, SU11657, SU5416, SU6668, Vatalanib (PTK-787/ZK222584), ZD6474, CP-547632, CEP-7055, CEP-5214, Bay43-9006, AZD217, PD0203359-0002, and GW654652; VEGF/VEGFR binding aptamers, for example, Mucagen® (pegaptanib; Eyetech/Pfizer Pharmaceuticals); inhibitors of VEGF/VEGFR gene expression, for example, antisense inhibitors of VEGF/VEGFR expression, anti-VEGF/VEGFR ribozymes, anti-VEGF/VEGFR siRNA, interferon, and thalidomide; miscellaneous VEGF/VEGFR inhibitors, for example, VEGF-Trap, VEGF-toxin conjugates, and anti-VEGF/VEGFR antibody-toxin conjugates; and endogenous angiogenesis inhibitors, for example, thrombospondin-1, angiostatin, 16-kDa prolactin fragment, interferon-α, interferon-β, interleukin-12, endostatin, tumstatin, SPARC, soluble Flt-1, kringle 5, AE-941, and vasohibin. See, e.g., Yang et al. (2004) Clin. Cancer Res. 10(18 Pt 2):6367S-70S; Susman et al. (2005) Lancet Oncol. 6:136; Jayson et al. (2005) Eur. J. Cancer 41:555-563; Zhang et al. (2002) Angiogenesis 5(1-2):35-44; Bergsland (2004) Am. J. Health-Syst Pharm 61:S4-S11; Rakhmilevich et al. (2004) Mol. Cancer. Ther. 3:969-976; Witte et al. (1998) Cancer Metastasis Rev. 17:155-161; Morimoto et al. (2004) Oncogene 23:1618-26; Backman et al. (2005) Pediatr. Res. February 17 (Epub ahead of print); Penland (2004) Clin. Colorectal Cancer 4 Suppl 2:S74-80; Tuccillo et al. Clin. (2005) Cancer Res. 11:1268-1276; Beebe et al. (2003) Cancer Res. 63:7301-7309; Gingrich et al. (2003) J. Med. Chem. 46:5375-5388; Ahmad et al. (2004) Clin. Cancer Res. 10(18 Pt 2):6388S-92S; Caponigro et al. (2005) Anticancer Drugs 16(2):211-221; Shi et al. (2004) Anticancer Res. 24:213-218; Ahmed et al. (2004) J. Chemother. 4:59-63; Clin. Lung Cancer (2004) 6:74-76; Huh et al. (2005) Oncogene 24:790-800; Hess-Stumpp et al. (2005) Chembiochem. 6:550-557; Riedel et al. (2005) Adv. Otorhinolaryngol. 62:103-20; Ciardiello et al. (2000) Clin. Cancer Res. 6:3739-3747; Shi et al. (2004) Anticancer Res. 24:213-218; Konner et al. (2004) Clin. Colorectal Cancer 4 Suppl 2:S81-85; Jin et al. (2002) Hum. Gene Ther. 13:497-508; Wild et al. (2000) Br. J. Cancer 83:1077-1083; Arora et al. (1999) Cancer Res. 59:183-188; Mayo et al. (2003) Am. J. Opthalmol. 136:1151-1152; Gottstein et al. (2001) Biotechniques 30(1)190-4, 196, 198 passim; Ciafre et al. (2004) J. Vasc. Res. 41:220-228; Gu et al. (2004) World J. Gastroenterol. 10: 1495-1498; Takano et al. (2004) Brain Tumor Pathol. 21:69-73; Colombel et al. (2005) Cancer Res. 65:300-308; Gao et al. (2002) J. Biol. Chem. 277:9492-9497; Kerbel et al. (2004) Clin. Invest. 114:884-886; Degeorges et al. (2004) Mol. Endocrinol. 8:2522-2542; Gordon (2004) Clin. Cancer Res. 10(18 Pt 2):6377S-63781S; Gollob et al. (2003) J. Clin. Oncol. 21:2564-2573; Rakhmilevich et al. (2004) Mol. Cancer. Ther. 3:969-976; Sund et al. (2005) Proc. Natl. Acad. Sci. U.S.A. 102:2934-2939; Chlenski et al. (2002) Cancer Res. 62:7357-7363; Gingras et al. (2001) Expert Rev Anticancer Ther. 1:341-347; Filleur et al. (2003) Cancer Res. 63:3919-3922; Amato (2005) Ann. Oncol. 16:7-15; Rini et al. (2005) J. Clin. Oncol. 23:1028-1043; the contents of which are herein incorporated by reference in their entireties. Alternatively, such agents can be contained in a separate composition from the VEGF-binding peptide and co-administered concurrently, before, or after the composition comprising a VEGF-binding peptide of the invention.

### D. Administration

At least one therapeutically effective cycle of treatment with a VEGF-binding peptide will be administered to a subject. By “therapeutically effective cycle of treatment” is intended a cycle of treatment that when administered, brings about a positive therapeutic response with respect to treatment of an individual for an angiogenesis disorder, cancer, or sepsis. Of particular interest is a cycle of treatment with a VEGF-binding peptide that prevents or delays the onset of symptoms or ameliorates symptoms of an angiogenesis disorder, cancer, or sepsis (e.g., suppresses angiogenesis, tumor growth, or infection). By “positive therapeutic response” is intended that the individual undergoing treatment according to the invention exhibits an improvement in one or more symptoms of an angiogenesis disorder, cancer, or sepsis, including such improvements as reduced blood vessel proliferation, neovascularization, and tumor growth, or reversal of infection and inflammation.

In certain embodiments, multiple therapeutically effective doses of compositions comprising one or more VEGF-binding peptides and/or one or more other therapeutic agents, such as VEGF/angiogenesis inhibitors, anti-cancer agents, anti-sepsis agents, or other medications will be administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, and so forth. For example, in some embodiments, one or more VEGF-binding peptides and/or one or more other therapeutic agents, such as VEGF/angiogenesis inhibitors, anti-cancer agents, anti-sepsis agents, or other medications, will be administered twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., twice-weekly or thrice-weekly administration of a therapeutically effective dose) for one or more weekly cycles until the desired therapeutic response is achieved. The agents can be administered by any acceptable route of administration as noted herein below.

The compositions of the present invention are typically, although not necessarily, administered orally, via injection (subcutaneously, intravenously or intramuscularly), by infusion, or locally. The pharmaceutical preparation can be in the form of a liquid solution or suspension immediately prior to administration, but may also take another form such as a syrup, cream, ointment, tablet, capsule, powder, gel, matrix, suppository, or the like. Additional modes of administration are also contemplated, such as pulmonary, rectal, transdermal, transmucosal, intrathecal, pericardial, intra-arterial, intracerebral, intraocular, intraperitoneal, and so forth. The pharmaceutical compositions comprising one or more VEGF-binding peptides and other agents may be administered using the same or different routes of administration in accordance with any medically acceptable method known in the art.

In a particular embodiment, a composition of the invention is used for localized delivery of a VEGF-binding peptide, for example, for the treatment of an angiogenesis disorder, cancer, or sepsis. The preparations according to the invention are also suitable for local treatment. For example, a VEGF-binding peptide may be administered by injection at the site of vascularization, tumor, or infection, or in the form of a solid, liquid, or ointment. Suppositories, capsules, in particular gastric-juice-resistant capsules, drops or sprays may also be used. The particular preparation and appropriate method of administration are chosen to target the site of the angiogenesis disorder, cancer, or infection.

In another embodiment, the pharmaceutical compositions comprising one or more VEGF-binding peptides and/or other agents are administered prophylactically, e.g., to prevent blood vessel proliferation or vascularization. Such prophylactic uses will be of particular value for subjects with known pre-existing angiogenesis disorders.

In another embodiment of the invention, the pharmaceutical compositions comprising one or more VEGF-binding peptides and/or other agents, are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The invention also provides a method for administering a conjugate comprising a VEGF-binding peptide as provided herein to a patient suffering from a condition that is responsive to treatment with a VEGF-binding peptide contained in the conjugate or composition. The method comprises administering, via any of the herein described modes, a therapeutically effective amount of the conjugate or drug delivery system, which may be provided as part of a pharmaceutical composition. The method of administering may be used to treat any condition that is responsive to treatment with a VEGF-binding peptide. More specifically, the compositions herein are effective in treating sepsis and angiogenesis disorders, including cancer, diabetic retinopathy, arthropathies, psoriasis and rheumatoid arthritis.

Those of ordinary skill in the art will appreciate which conditions a specific VEGF-binding peptide can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

Generally, a therapeutically effective amount will range from about 0.50 mg to 5 grams of a VEGF-binding peptide daily, in some cases, from about 5 mg to 2 grams daily, and in other cases, from about 7 mg to 1.5 grams daily. In certain embodiments, such doses are in the range of 10-600 mg four times a day (QID), 200-500 mg QID, 25-600 mg three times a day (TID), 25-50 mg TID, 50-100 mg TID, 50-200 mg TID, 300-600 mg TID, 200-400 mg TID, 200-600 mg TID, 100 to 700 mg twice daily (BID), 100-600 mg BID, 200-500 mg BID, or 200-300 mg BID. The amount of compound administered will depend on the potency of the specific VEGF-binding peptide and the magnitude or effect on angiogenesis desired and the route of administration.

A purified VEGF-binding peptide (which may be provided as part of a pharmaceutical preparation) can be administered alone or in combination with other VEGF-binding peptides or therapeutic agents, such as VEGF/angiogenesis inhibitors, anti-cancer agents, anti-sepsis agents, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. In one embodiment, compositions require dosing no more than once a day.

A VEGF-binding peptide can be administered prior to, concurrent with, or subsequent to other agents. If provided at the same time as other agents, one or more VEGF-binding peptides can be provided in the same or in a different composition. Thus, one or more VEGF-binding peptides and other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising a VEGF-binding peptide and a dose of a pharmaceutical composition comprising at least one other agent, such as a VEGF/angiogenesis inhibitor, anti-cancer agent, or anti-sepsis agent, which in combination comprise a therapeutically effective dose, according to a particular dosing regimen. Similarly, one or more VEGF-binding peptides and one or more other therapeutic agents can be administered in at least one therapeutic dose. Administration of the separate pharmaceutical compositions can be performed simultaneously or at different times (i.e., sequentially, in either order, on the same day, or on different days), so long as the therapeutic effect of the combination of these substances is caused in the subject undergoing therapy.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Screening of Libraries for High Affinity VEGF-Binding Peptides

To identify novel high affinity VEGF binding peptides, a focused VEGF-binding library (X₆-W-E/D-W-E/D-X₉) was constructed and screened using bacterial display. The sorting procedure for this library employed low concentrations of VEGF (1-10 nM) for the first rounds and a 15-minute dissociation time for the final round. All sorting (beginning with F1) was performed in the presence of 0.075% (w/v) BSA. This concentration corresponds to ˜1/100 dilution of serum (total protein concentration 6.3-8.6% w/v).

Twenty-seven independent sequences were isolated from the third and fourth rounds of sorting. These were assayed for binding to VEGF at 1 nM and 10 nM, dissociation rate at room temperature, and retention of binding in the presence of serum. Two sequences were chosen to move forward with based on their slow dissociation rate: CPVQTMWDWECMRAFIEG (L2.F3B.08) and NFGYGKWEWDYGKWLEKVG (L2.F4.04), and 15-mer libraries have been constructed on the N-terminus of each clone. The libraries contain 4×10⁷ and 1×10⁸ members, respectively.

### Library Screening

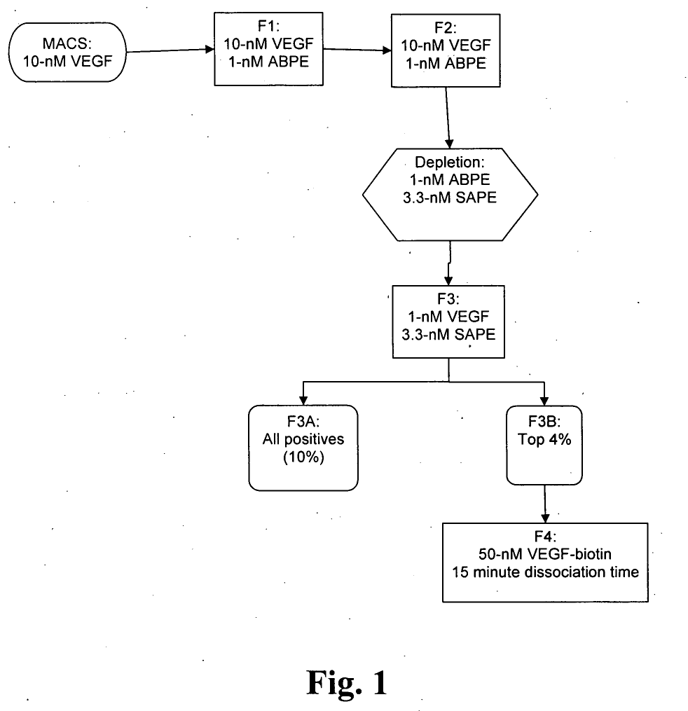

The focused VEGF-binding library, consisting of 2×10⁸ members of the form X₆-W-E/D-W-E/D-X₉, was initially pre-enriched by MACS before beginning screening by FACS. The successful library screening scheme is described below and summarized in FIG. 1.

After MACS with 10-nM VEGF, the resulting population was labeled with 10-nM VEGF in 0.075% BSA for the first two rounds of FACS sorting. The secondary label in both these rounds was antibiotin-phycoerythrin (ABPE). In the post-sort scanning of the F2 population, ˜3% of this pool bound SAPE, ABPE, or both. As a result, an intermediate depletion step was performed in which the F2 population was labeled with SAPE and ABPE, and unlabeled (dark) cells were isolated and saved for future sorting. For the third round of FACS, the depleted F2 population was labeled with 1-nM VEGF. Secondary labeling was performed with SAPE. At this concentration, ˜10% of the cells were labeled as positive. Two populations were saved as F3-one in which all positive events were retained (F3A) and one that saved the top 4% (F3B), which corresponded to the top 10% of the positive control.

The F3B population was further sorted using a kinetic sorting procedure. This population was incubated with 50-nM VEGF, a concentration at which ˜65% of the population was fluorescent above background. The excess VEGF was washed away, and the cells were diluted 1/10 in PBS. The VEGF was allowed to dissociate for 5, 10, and 15 minutes at room temperature. After this time, SAPE was added for secondary labeling on ice. After 15 minutes, ˜58% of the population was still labeled positive. A sort gate was drawn to encompass the top 10% of the positive control. At t=0, this gate encompassed ˜30% of the F3B population. At t=15 minutes, this had reduced to 10%, and by the time sorting could be performed (˜8 minutes later), this had reduced to 3%.

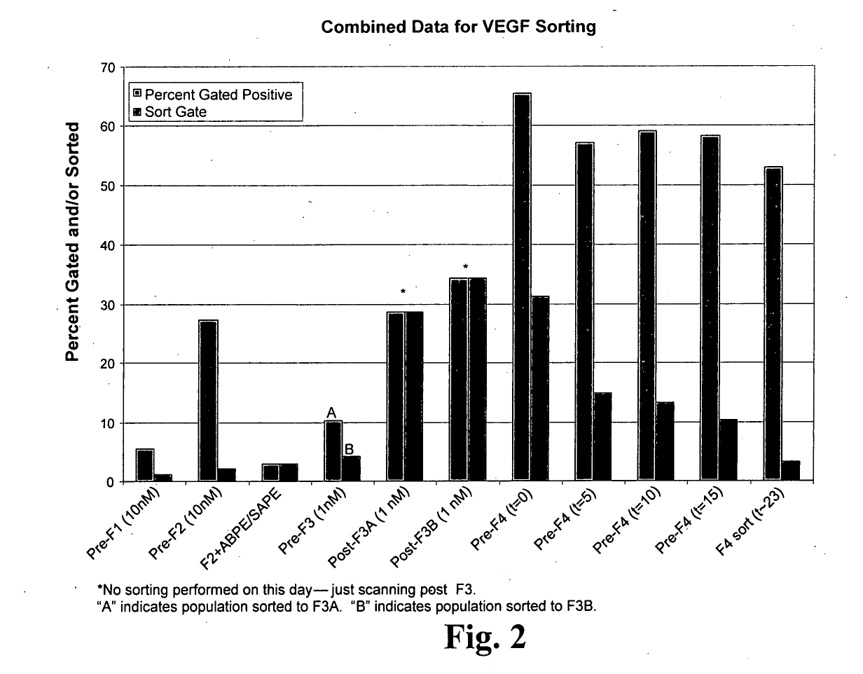

FIG. 2 shows the enrichment data for these rounds of sorting. Data are shown for the portion of each population considered positively labeled and the portion that was sorted. The concentration of VEGF used for F1 through F3 is given in parentheses. Only the 15-minute dissociation time was sorted, but all other time points are given for reference. “F4 sort” refers to the fraction gated immediately before and during the sorting process.

### Clone Characterization

Individual clones were selected from the F3A, F3B, and F4 populations and analyzed for their equilibrium binding to VEGF and dissociation rate. In total, twenty-seven individual sequences were isolated. Table 3 shows the sequences in numerical order, where L2 indicates the W-E/D-W-E/D library and F# indicates the round from which the clone was isolated. Three consensus groups appeared (shown in Table 4): two which included WWL upstream of W-E/D-W-E/D and one set of disulfide constrained peptides which resembled the Genentech v114 peptide. In addition, nearly all the sequences showed a preference for positively charged amino acids downstream of the fixed W-E/D-W-E/D portion.

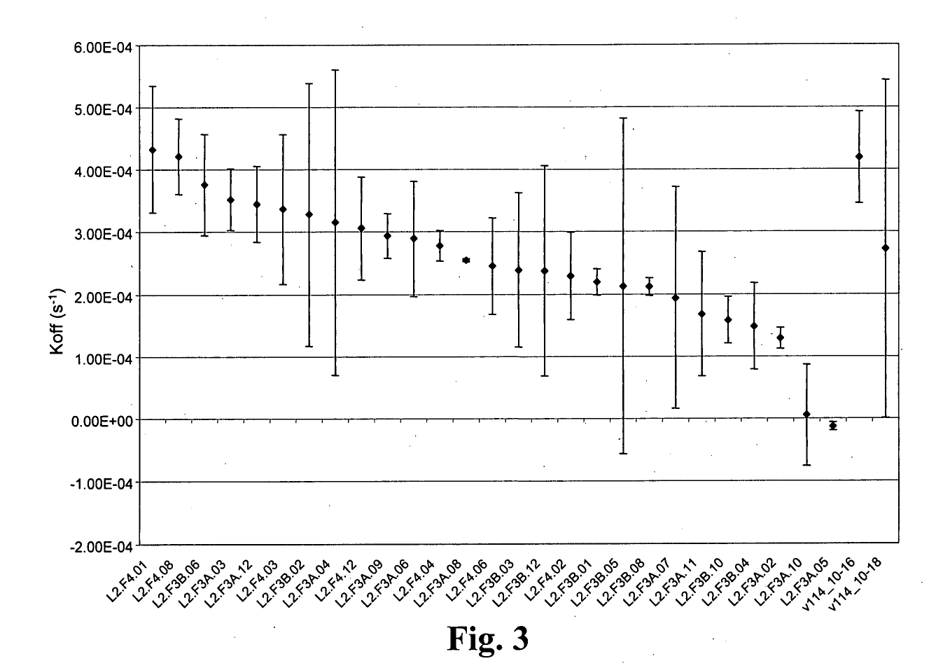

Equilibrium binding with VEGF at 1 nM and 10 nM (and 0.075% BSA) indicated that a majority of the clones had good binding affinity and/or high peptide expression. Initial measurements of off-rate were made for each clone in order to narrow down the search for the best binding peptide. Each clone was labeled with 10-nM VEGF. Excess VEGF was washed away, and the protein was allowed to dissociate for 15 and 30 minutes at room temperature. SAPE was added, and secondary labeling was performed on ice. Excel's Regression function was used to calculate k_{off} for each peptide using three time points (0, 15, and 30 minutes) and the standard error for the regression. This data is shown in FIG. 3, ranked by k_{off}, with the results for v114 on two different days given last.

Based on off-rate and display, six clones were chosen for further characterization and are indicated in bold in Table 3. L2.F3A.02 and L2.F3A.10 both contain a WWL sequence, and although L2.F3A.02 showed less error in off-rate calculations, L2.F3A.10 had better labeling. L2.F3B.08 was chosen as a representative v114-like clone with good dissociation kinetics. L2.F3A.05, L2.F3B.12, and L2.F4.04 also had slow dissociation rates and did not share any common sequence elements with the other chosen clones. Furthermore, L2.F4.04 was the most common sequence isolated.

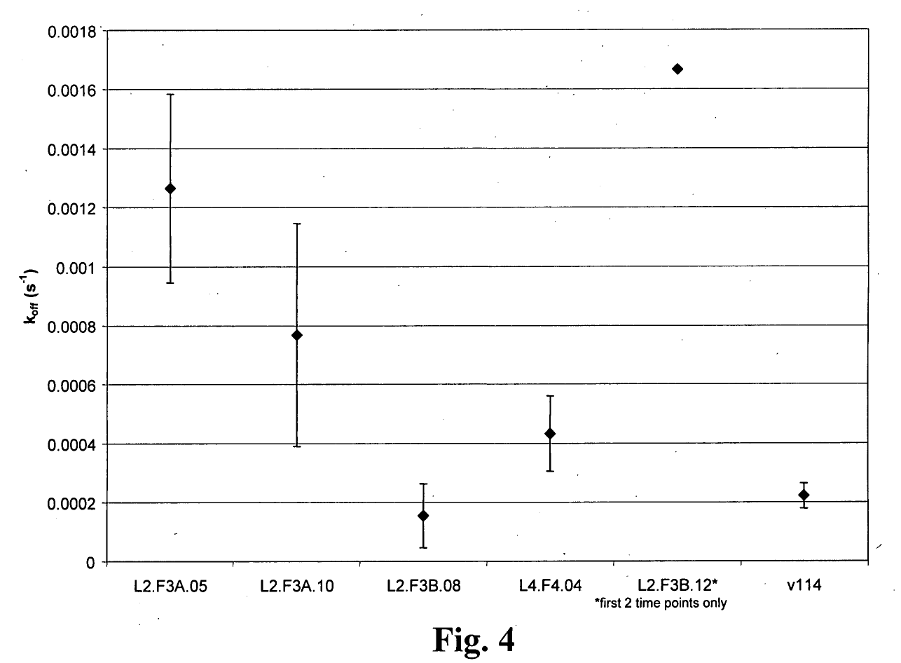

Each clone was incubated with 10 nM VEGF and allowed to come to equilibrium. The excess VEGF was washed away, and the cells were separated into 4 aliquots and diluted 1/10 in room temperature PBS. SAPE was added to the appropriate aliquot after 5, 10, 15, and 30 minutes for secondary labeling on ice. The mean fluorescence at these time points was used to determine k_{off}. In most cases, the t=30 minute point could not be incorporated into the regression. FIG. 4 shows the k_{off} with the standard error of regression for the remaining five clones.

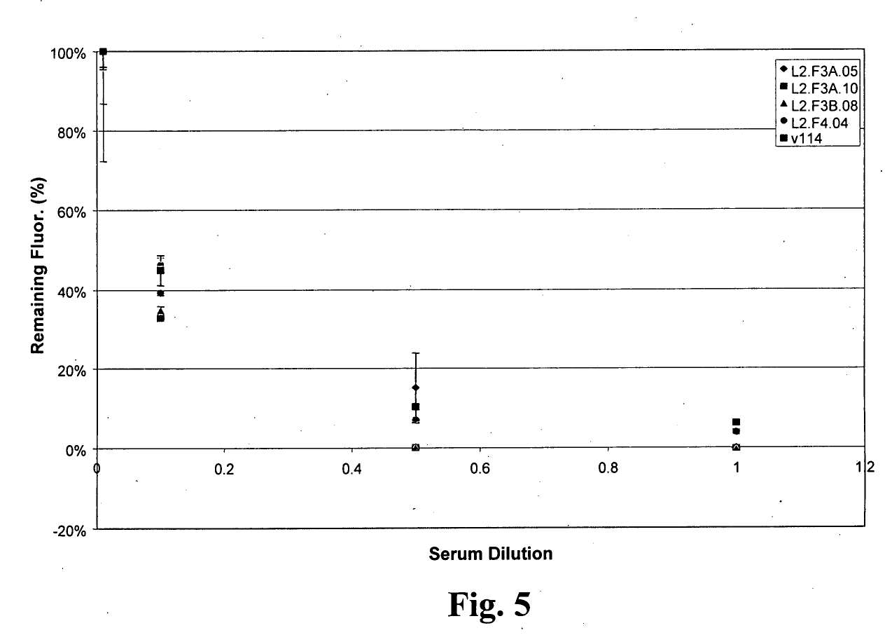

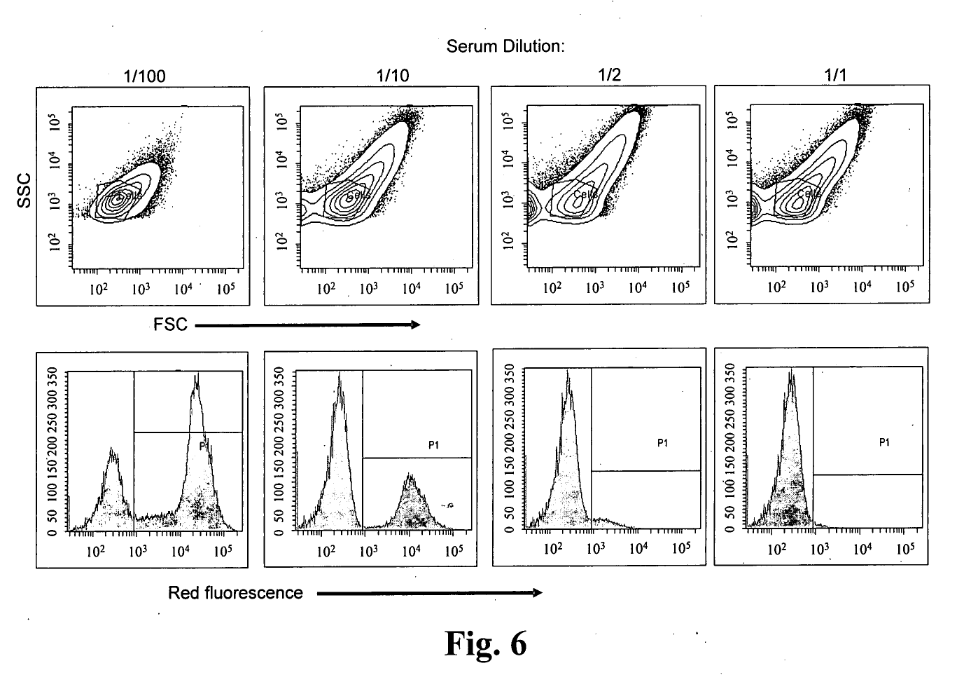

In addition, measurements of specificity were performed for the four peptides whose off-rate could be calculated. Each clone was incubated with 10-nM VEGF in varying dilutions of BSA and serum followed by secondary labeling with SAPE at the same BSA/serum concentration. It was assumed that a specific peptide would retain more fluorescence upon serum addition than a non-specific peptide. The serum was first prepared by incubation with cells overexpressing the CPXopt template, spinning down the cell pellet and any bacteria-specific proteins bound to them, and removing the supernatant for incubation with the selected clones. Adding BSA up to physiological conditions decreased the mean fluorescence of each clone by an average of ˜40%, seemingly indicating some degree of specificity. Serum, on the other hand, appeared to abolish binding completely. FIG. 5 gives the fluorescence remaining after serum addition relative to initial fluorescence (at a serum dilution of 1/100), which was taken to be a measure of the specificity of the interaction between the peptide and VEGF. As shown in FIG. 6, which gives sample histograms for one replicate of L2.F4.04, the addition of serum created an additional negative population of cell-sized protein aggregates that appeared in all fluorescence histograms. Therefore, the change in fluorescence of a gated population of positive binding cells was used to determine the specificity metric. In some cases with undiluted serum, a significant fraction of cells (>˜1%) still fell into this positive, and the fluorescence of this small fraction of cells was used to calculate the specificity. In other cases, the final data point was calculated using the mean fluorescence of the entire population. Even with relatively small error from these duplicate runs, it is difficult to interpret the value and validity of these data, as all the peptides seem to fall on the same curve.

### Construction of a Bidentate Library

The choice of optimum peptide sequence from this biased library was based on dissociation rate with some respect given to display level and specificity. As shown in FIG. 4, two peptides-L2.F3B.08 (CPVQTMWDWECMRAFIEG, SEQ ID NO:18) and L2.F4.04 (NFGYGKWEWDYGKWLEKVG, SEQ ID NO:24)—have dissociation rates similar to or slower than the v114 positive control. Moreover, both of these peptides display well on the surface of the cell and give bright fluorescence signals when bound to VEGF. In looking at the apparent specificity, L2.F3B.08 loses more fluorescence upon serum addition than does L2.F4.04. Thus, both peptides were used to design bidentate libraries.

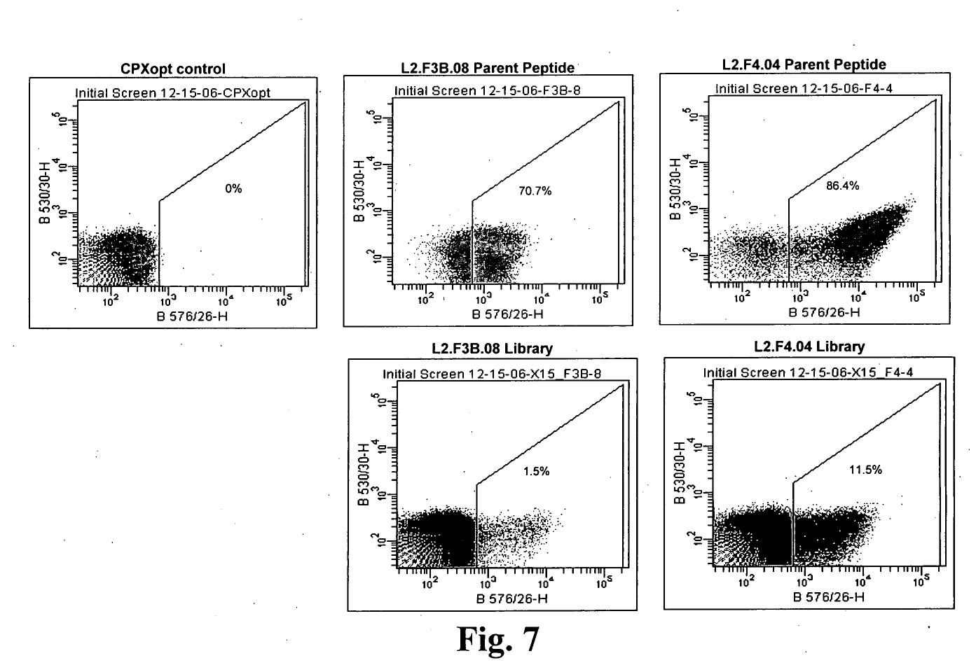

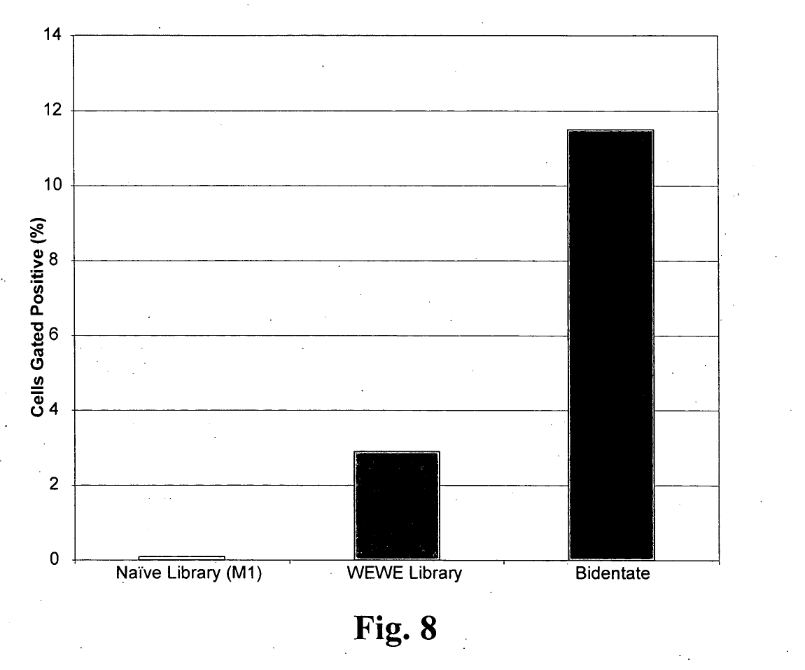

Two libraries were constructed, one based on each of the above peptides, in which a random 15-mer was placed upstream of the peptide on CPXopt. A seven-amino acid linking sequence of GGGSGGG was inserted between the fixed peptide and the random portion. The size of each library was intentionally kept small so that all screening could be done by FACS, without the need of any pre-enrichment step. In the end, the L2.F4.04 library had 1×10⁸ members, and the L2.F3B.08 library had 4×10⁷ members. Each library was incubated with 10-nM VEGF in the presence of BSA, as before, to check for binding. As shown in FIG. 7, there appears to be a good proportion of peptides that already bind VEGF and that display well on the cell surface. Moreover, FIG. 8 gives the change in percent positive events with each round of library creation. The “naïve” library indicates the CPX-X₁₅ library after one round of MACS with VEGF (˜0.1% labeled). Data for the WEWE focused library and L2.F4.04 bidentate library are given without any pre-enrichment steps.

TABLE 3

Independent sequences isolated from FACS
rounds 3 and 4

| | | | |
|---|---|---|---|
| SEQ | | | Number of |
| ID | | | Occurrences |
| NO | Clone | Sequence | (if >1) |

| | | | |
|---|---|---|---|
| 1 | L2.F3A.02 | SLGWWLWDWEKQVQLDRRK | |
| 2 | L2.F3A.03 | AWWLQRWDWEDRQGERGMW | |
| 3 | L2.F3A.04 | MGNKQGWEWDYFRWVQMAA | |
| 4 | L2.F3A.05 | HQQLQYWDWIEAVVHNG | |
| 5 | L2.F3A.06 | AAGWWLWDWEAGRQLRDRL | |
| 6 | L2.F3A.07 | RACMGGWEWDYWRAMSVGH | |
| 7 | L2.F3A.08 | VWHQVPWDWEMLLGKNLDR | |
| 8 | L2.F3A.09 | YSVSRMWDWEKVKWVPWPA | |
| 9 | L2.F3A.10 | GPGWWLWDWDNLGARRGGL | |
| 10 | L2.F3A.11 | GAALTRWEWEVYKLVENCM | 2 |
| 11 | L2.F3A.12 | GWWLNSWDWEHNTSLGPGV | |
| 12 | L2.F3B.O1 | LLGWWLWDWDGARSMNGRW | |
| 13 | L2.F3B.02 | WMGWSTWEWDRGGMMRSST | |
| 14 | L2.F3B.03 | AWWLRTWDWERSNH | |
| 15 | L2.F3B.04 | PGGWWLWDWERVRGGEHLR | |
| 16 | L2.F3B.05 | DKPWWLWDWEKGQVGSSRS | |
| 17 | L2.F3B.06 | GESWWLWDWDWGSKRQLVA | |
| 18 | L2.F3B.08 | CPVQTMWDWECMRAFIEG | |
| 19 | L2.F3B.10 | GWWLSTWEWERSALAAEQK | |
| 20 | L2.F3B.12 | WKELRFWDWEGGNHKKCVT | |
| 21 | L2.F4.01 | WKELRFWDWEGGNHKKCVT | 2 |
| 22 | L2.F4.02 | WRLLPTWDWDWGQSSTETM | |
| 23 | L2.F4.03 | WCPLSGWDWEGSVCRSGGS | |
| 24 | L2.F4.04 | NFGYGKWEWDYGKWLEKVG | 7 |
| 25 | L2.F4.06 | CLRQIIWDWECFRTNNTMV | 2 |
| 26 | L2.F4.08 | WIPLQLWDWERDSCSTIGL | |
| 27 | L2.F4.12 | YVNLWGWEGWGGQSEQ | |

Note:
SEQ ID NOS:18, 23, and 25 are sequences of disulfide constrained peptides.
SEQ ID NO:27 contains the altered linker sequence GGQSEQ.

TABLE 4
Consensus motifs

| | | |
|---|---|---|
| SEQ | | |
| ID | | |
| NO | Clone | Sequence |

| | | |
|---|---|---|
| 1 | L2.F3A.02 | SLGWWLWDWEKQVQLDRRK |
| 5 | L2.F3A.06 | AAGWWLWDWEAGRQLRDRL |
| 9 | L2.F3A.10 | GPGWWLWDWDNLGARRGGL |
| 17 | L2.F3B.06 | GESWWLWDWDWGSKRQLVA |
| 12 | L2.F3B.01 | LLGWWLWDWDGARSNNGRW |
| 15 | L2.F3B.04 | PGGWWLWDWERVRGGEHLR |
| 16 | L2.F3B.05 | DKPWWLWDWEKGQVGSSRS |
| 28 | Consensus | GWWLWDWE G R |
| 2 | L2.F3A.03 | AWWLQRWDWEDRQGERGMW |
| 11 | L2.F3A.12 | GWWLNSWDWEHNTSLGPGV |
| 14 | L2.F3B.03 | AWWLRTWDWERSNH----- |
| 19 | L2.F3B.10 | GWWLSTWEWERSALAAEQK |
| 29 | Consensus | AWWLNTWDWERSN |
| 31 | v114 | VEPNCDIHVMWEWECFERL |
| 18 | L2.F3B.08 | ----CPVQTMWDWECMRAFIEG |
| 25 | L2.F4.06 | ----CLRQIIWDWECFRTNNTMV |
| 30 | Consensus | C IQIMWDWECFR |

### CONCLUSION

A focused W-E/D-W-E/D VEGF-binding library was successfully screened to yield peptides with high affinity for VEGF. Out of twenty-seven clones isolated from the third and fourth round of FACS screening, two were selected for the basis of a new bidentate library. The new libraries consisted of fifteen random amino acids followed by a GGGSGGG linker upstream of the selected peptides on CPXopt. Initial screening of each library indicated that a large fraction of the bidentate library bound to VEGF. This library was further screened, as described below, for sequences that bound with increased affinity and specificity for VEGF.

### Example 2

### Characterization of Bidendate VEGF-Binding Peptides

### Measurement of Dissociation Rate Constants

First order dissociation rate constants (k_{off}) were measured for each peptide when displayed on the cell surface at room temperature and also at 37° C. Cells displaying VEGF-binding peptides were first incubated with biotinylated VEGF and allowed to equilibrate at 4° C. After centrifugation, excess VEGF was removed. The cells were then resuspended in buffer, such that the concentration of cells was diluted 1:10, as compared to the VEGF-labeling step. Cells were allowed to incubate in buffer at room temperature or 37° C. for times varying from 5 minutes to 21 hours to allow dissociation of VEGF from the cell surface. After the appropriate amount of time, the cells were placed on ice to stop the dissociation, and streptavidin-phycoerythrin (SAPE) was added for fluorescence labeling. After centrifugation, excess SAPE was removed, and cellular fluorescence was measured by flow cytometry. Fluorescence data as a function of dissociation time was used to determine k_{off}. These data as well as apparent equilibrium dissociation constants (K_{D}) are given in Table 5.

Competitive Inhibition with Soluble Peptides

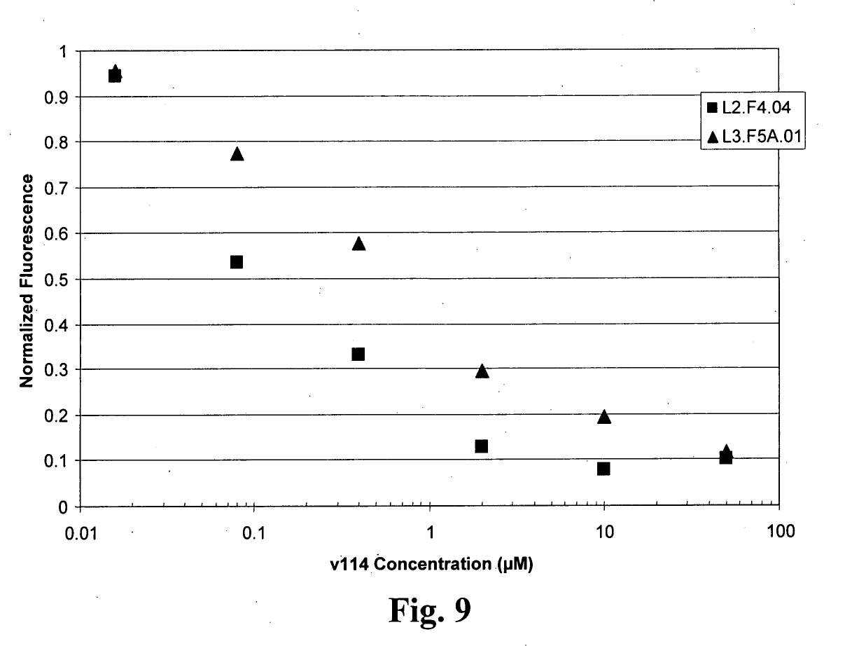

The inhibition of VEGF binding to the displayed VEGF-binding peptides by a previously isolated VEGF-binding peptide v114 (VEPNCDIHVMWEWECFERL; SEQ ID NO:31) was determined. The reported equilibrium dissociation constant (K_{D}) of V114 is 110 nM (Fairbrother et al. (1998) Biochemistry 37:17754-17764; herein incorporated by reference). Biotinylated VEGF was first mixed with various concentrations of soluble v114 peptide (Anaspec); the ratio of soluble peptide to VEGF ranged from ˜2:1 to 5000:1. Cells expressing VEGF binding peptides were then incubated with this VEGF/peptide mixture and allowed to equilibrate at 4° C. After centrifugation, unbound protein/peptide was removed, and the cells were resuspended in SAPE for fluorescence labeling. Decreased cellular fluorescence (as measured by flow cytometry) upon increased v114 peptide concentration was indicative of inhibition of VEGF binding to the cell surface-displayed peptide. Since v114 is known to bind the receptor binding-pocket of VEGF, it is infered that peptides competitively inhibited by v114 also bind this same region. There appears to be at least a 5-fold increase in the concentration of v114 required to block 50% of VEGF binding (decrease fluorescence by 50%) for the bidentate peptide as compared to the parent peptide alone which implies a significant increase in the affinity of the bidentate peptide for VEGF. These results are shown in FIG. 9.

### Murine Cross-Reactivity

Cells expressing VEGF-binding peptides were incubated with biotinylated recombinant murine VEGF (rmVEGF). Fluorescence labeling was performed with SAPE as previously described. The first seven clones listed in Table 5 as well as v114 were assayed for cross-reactivity with rmVEGF. All of the ligands analyzed bound rmVEGF at concentrations of 25 and 250 nM, as measured by flow cytometry. These results indicate that the peptides of the invention are suitable for direct testing in mouse models.

TABLE 5

Parent and bidentate VEGF-binding peptides and first order dissociation
rate constants (k_{off}) and apparent equilibrium dissociation constants (K_{D}),
as measured from the bacterial cell surface

| | | | | |
|---|---|---|---|---|
| SEQ | | | | |
| ID | Sequence | RT k_{off} | 37° C. k_{off} | KD |

| | | | | | | | |
|---|---|---|---|---|---|---|---|
| NO | Clone | Peptide 1 | Linker | Peptide 2 | (s⁻¹) | (s⁻¹) | (nM) |

| | | | | | | | |
|---|---|---|---|---|---|---|---|
| 18 | L2.F3B.08 | | | CPVQTMWDWECMRAFIEG | 7.2E−03 | 4.2E−03 | 17.8 |
| 24 | L2.F4.04 | | | NFGYGKWEWDYGKWLEKVG | 1.3E−03 | 1.3E−03 | 6.1 |
| 32 | L3.F5A.01 | SSKCGRWGETPCLAE | GGGSGGG | NFGYGKWEWDYGKWLEKVG | 2.0E−05 | 1.2E−04 | 2.1 |
| 33 | L3.F5A.02 | VTSRYEMREDGSWKK | GGGSGGG | NFGYGKWEWDYGKWLEKVG | ~0 | 7.4E−04 | 2.6 |
| 34 | L3.F5A.03 | AKRSSYSMWGAMP | GGGSGGG | NFGYGKWEWDYGKWLEKV | 4.28E−05 | 6.0E−04 | 3.7 |
| 35 | L3.F6.01 | SYVLKEWSVPSWGKP | GGGSGGG | NFGYGKWEWDYGKWLEKV | 1.10E−04 | 2.5E−04 | 3.5 |
| 36 | L3.F6.02 | SKLKTLLWQQ | GGGSGGG | NFGYGKWEWDYGKWLEKV | 2.91E−04 | 1.4E−03 | 5.0 |
| 37 | L3B.F4A.02 | LLQKVMAXAGRGLPR | GGGSGGG | CPVQTMWDWECMRAFIEG | | | |
| 38 | L3B.F4A.05 | GQSLA | GGGSGGG | CPVQTMWDWECMRAFIEG | | | |
| 39 | L3B.F4B.02 | GQSL | GGGSGGG | CPVQTMWDWECMRAFIEG | | | |
| 40 | L3B.F4B.05 | ALSKATEGVLRSLRG | GGGSGGG | CPVQTMWDWECMRAFIEG | | | |
| 41 | L3B.F4B.06 | LLRVLTAAVGSSRGV | GGGSGGG | CPVQTMWDWECMRAFIEG | | | |

Sequences whose names begin with L2 indicate peptides with single binding interactions.
Sequences whose names begin with L3 are deemed “bidentate” and represent two joined peptide motifs that work cooperatively to bind VEGF.

Based on a comparison of the dissociation rates at room temperature, as shown in Table 5, the bidentate peptides exhibit about a 4 to 65-fold improvement (slower dissociation) at room temperature and up to a 12-fold improvement at 37° C. as compared to the monodendate parent peptide L2.F4.04.

Thus, novel VEGF-binding peptides are disclosed. Although preferred embodiments of the subject invention have been described in some detail, it is understood that obvious variations can be made without departing from the spirit and the scope of the invention as described herein.

1. The peptide according to claim 3, comprising an amino acid sequence selected from the group consisting of SEQ ID NOS: 1-30 and 32-51.

2. The peptide of claim 1 consisting of an amino acid sequence selected from the group consisting of SEQ ID NOS:1-30 and 32-51.

3. A peptide comprising a W-E/D-W-E/D motif that binds to vascular endothelial growth factor (VEGF).

4. The peptide of claim 3, wherein said peptide binds to VEGF with a dissociation constant (K_{D}) of less than 10⁻⁶ M.

5. The peptide of claim 4, wherein said peptide binds to VEGF with a dissociation constant (K_{D}) of less than 10⁻⁸ M.

6. The peptide of claim 5, wherein said peptide binds to VEGF with a dissociation constant (K_{D}) of less than 10⁻¹⁰ M.

7. The peptide of claim 6, wherein said peptide binds to VEGF with a dissociation constant (K_{D}) of less than 10⁻¹² M.

8. The peptide of claim 3 comprising a sequence selected from the group consisting of SEQ ID NOS:28-30.

9. The peptide of claim 3 comprising an amino acid sequence selected from the group consisting of SEQ ID NOS: 1-30 and 32-51.

10. The peptide of claim 3 comprising the amino acid sequence of SEQ ID NO:18.

11. The peptide of claim 3 comprising the amino acid sequence of SEQ ID NO:24.

12. The peptide of claim 3, wherein the peptide is a bidentate peptide comprising a first VEGF-binding peptide sequence and a second VEGF-binding peptide sequence.

13. The peptide of claim 12, wherein the first peptide sequence comprises a sequence selected from the group consisting of SEQ ID NOS: 1-31, and the second peptide sequence comprises a sequence selected from the group consisting of SEQ ID NOS:42-51.

14. The peptide of claim 13, wherein the bidentate peptide comprises a sequence selected from the group consisting of SEQ ID NOS:32-41.

15. The peptide of claim 12, wherein the first and second VEGF-binding peptides are joined by a linker of two or more residues.

16. The peptide of claim 15, wherein the linker comprises the sequence of SEQ ID NO:52.

17. A composition comprising at least one peptide according to claim 3.

18. The composition of claim 17, further comprising a pharmaceutically acceptable excipient.

19. The composition of claim 17, further comprising a vascular endothelial growth factor (VEGF)/angiogenesis inhibitor.

20. The composition of claim 19, wherein the VEGF/angiogenesis inhibitor is selected from the group consisting of an anti-VEGF/VEGFR antibody, a VEGF receptor tyrosine kinase inhibitor, a VEGF/VEGFR binding aptamer, an antisense inhibitor of VEGF/VEGFR expression, an anti-VEGF/VEGFR ribozyme, an anti-VEGF/VEGFR siRNA, and an endogenous VEGF/angiogenesis inhibitor.

21. The composition of claim 17, further comprising an anti-cancer agent.

22. The composition of claim 17, further comprising an anti-sepsis agent.

23. A method of identifying a peptide comprising a W-E/D-W-E/D motif that binds to VEGF, the method comprising:
a) providing a library of peptides wherein each peptide in the library comprises the W-E/D-W-E/D motif; and
b) screening the library for peptides that bind to VEGF.

24. The method of claim 23, wherein the library is screened for peptides that bind to VEGF by sequential magnetic-activated cell sorting (MACS).

25. The method of claim 24, further comprising screening the library for peptides that bind to VEGF by fluorescence-activated cell sorting (FACS).

26. A method of treating a disorder selected from the group consisting of an angiogenesis disorder, cancer, and sepsis, the method comprising administering to a subject a therapeutically effective amount of the composition of claim 17.

27. The method of claim 26, wherein the peptide is administered prophylactically.

28. The method of claim 26, wherein administration of the peptide delays the onset of one or more symptoms of the disorder.

29. The method of claim 26, wherein administration of the peptide inhibits angiogenesis.

30. The method of claim 26, wherein administration of the peptide inhibits tumor growth.

31. The method of claim 26, wherein administration of the peptide inhibits infection.

32. A method for modulating angiogenesis in a subject comprising administering a therapeutically effective amount of the composition of claim 17.

33. The method of claim 32, wherein the composition inhibits angiogenesis in the subject.

34. The method of claim 32, wherein the composition is administered intermittently.

35. The method of claim 33, wherein the method further comprises administering to the subject a therapeutically effective amount of a vascular endothelial growth factor (VEGF)/angiogenesis inhibitor.

36. The method of claim 35, wherein the VEGF/angiogenesis inhibitor is selected from the group consisting of an anti-VEGF/VEGFR antibody, a VEGF receptor tyrosine kinase inhibitor, a VEGF/VEGFR binding aptamer, an antisense inhibitor of VEGF/VEGFR expression, an anti-VEGF/VEGFR ribozyme, an anti-VEGF/VEGFR siRNA, and an endogenous VEGF/angiogenesis inhibitor.

37. A method for inhibiting VEGF activity in a subject comprising administering a therapeutically effective amount of the composition of claim 17.

38. The method of claim 37, wherein the subject has cancer.

39. The method of claim 38, wherein the method further comprises administering to the subject a therapeutically effective amount of an anti-cancer agent.

40. The method claim 37, wherein the subject has sepsis.

41. The method of claim 40, wherein the method further comprises administering to the subject a therapeutically effective amount of an anti-sepsis agent.

42. The method of claim 41, wherein the composition is administered intermittently.

43. A method for detecting the presence of VEGF in a biological sample, the method comprising:
a) exposing the biological sample suspected of containing VEGF to the peptide of claim 3; and
b) detecting the presence or absence of the peptide bound to the VEGF, if any, in the sample.

44. The method of claim 43, wherein the peptide comprises a detectable label.

45. The method of claim 43, wherein the biological sample is obtained from a subject who has an angiogenesis disorder, cancer, or sepsis.

46. The method of claim 45, further comprising comparing the amount of VEGF in the biological sample from the subject who has an angiogenesis disorder, cancer or sepsis to the amount of VEGF in a corresponding biological sample from a normal subject.

47. The method of claim 43, further comprising collecting a plurality of biological samples from a subject at different time points and comparing the amount of VEGF in each biological sample to determine if the amount of VEGF is increasing or decreasing in the subject over time.

48. A complex comprising the peptide of claim 3 and VEGF.

49. The complex of claim 48 comprising an amino acid sequence selected from the group consisting of SEQ ID NOS:1-30 and 32-51.


SEQUENCE LISTING (52 sequences)

SEQ ID NO: 1 - protein, 19 aa, Artificial
  VEGF-binding peptide
SLGWWLWDWEKQVQLDRRK

SEQ ID NO: 2 - protein, 19 aa, Artificial
  VEGF-binding peptide
AWWLQRWDWEDRQGERGMW

SEQ ID NO: 3 - protein, 19 aa, Artificial
  VEGF-binding peptide
MGNKQGWEWDYFRWVQMAA

SEQ ID NO: 4 - protein, 17 aa, Artificial
  VEGF-binding peptide
HQQLQYWDWIEAVVHNG

SEQ ID NO: 5 - protein, 19 aa, Artificial
  VEGF-binding peptide
AAGWWLWDWEAGRQLRDRL

SEQ ID NO: 6 - protein, 19 aa, Artificial
  VEGF-binding peptide
RACMGGWEWDYWRAMSVGH

SEQ ID NO: 7 - protein, 19 aa, Artificial
  VEGF-binding peptide
VWHQVPWDWEMLLGKNLDR

SEQ ID NO: 8 - protein, 19 aa, Artificial
  VEGF-binding peptide
YSVSRMWDWEKVKWVPWPA

SEQ ID NO: 9 - protein, 19 aa, Artificial
  VEGF-binding peptide
GPGWWLWDWDNLGARRGGL

SEQ ID NO: 10 - protein, 19 aa, Artificial
  VEGF-binding peptide
GAALTRWEWEVYKLVENCM

SEQ ID NO: 11 - protein, 19 aa, Artificial
  VEGF-binding peptide
GWWLNSWDWEHNTSLGPGV

SEQ ID NO: 12 - protein, 19 aa, Artificial
  VEGF-binding peptide
LLGWWLWDWDGARSMNGRW

SEQ ID NO: 13 - protein, 19 aa, Artificial
  VEGF-binding peptide
WMGWSTWEWDRGGMMRSST

SEQ ID NO: 14 - protein, 14 aa, Artificial
  VEGF-binding peptide
AWWLRTWDWERSNH

SEQ ID NO: 15 - protein, 19 aa, Artificial
  VEGF-binding peptide
PGGWWLWDWERVRGGEHLR

SEQ ID NO: 16 - protein, 19 aa, Artificial
  VEGF-binding peptide
DKPWWLWDWEKGQVGSSRS

SEQ ID NO: 17 - protein, 19 aa, Artificial
  VEGF-binding peptide
GESWWLWDWDWGSKRQLVA

SEQ ID NO: 18 - protein, 18 aa, Artificial
  VEGF-binding peptide
CPVQTMWDWECMRAFIEG

SEQ ID NO: 19 - protein, 19 aa, Artificial
  VEGF-binding peptide
GWWLSTWEWERSALAAEQK

SEQ ID NO: 20 - protein, 19 aa, Artificial
  VEGF-binding peptide
WKELRFWDWEGGNHKKCVT

SEQ ID NO: 21 - protein, 19 aa, Artificial
  VEGF-binding peptide
WKELRFWDWEGGNHKKCVT

SEQ ID NO: 22 - protein, 19 aa, Artificial
  VEGF-binding peptide
WRLLPTWDWDWGQSSTETM

SEQ ID NO: 23 - protein, 19 aa, Artificial
  VEGF-binding peptide
WCPLSGWDWEGSVCRSGGS

SEQ ID NO: 24 - protein, 19 aa, Artificial
  VEGF-binding peptide
NFGYGKWEWDYGKWLEKVG

SEQ ID NO: 25 - protein, 19 aa, Artificial
  VEGF-binding peptide
CLRQIIWDWECFRTNNTMV

SEQ ID NO: 26 - protein, 19 aa, Artificial
  VEGF-binding peptide
WIPLQLWDWERDSCSTIGL

SEQ ID NO: 27 - protein, 16 aa, Artificial
  VEGF-binding peptide
YVNLWGWEGWGGQSEQ

SEQ ID NO: 28 - protein, 16 aa, Artificial
  VEGF-binding peptide
GWWLWDWEXGXXXXXR

SEQ ID NO: 29 - protein, 13 aa, Artificial
  VEGF-binding peptide
AWWLNTWDWERSN

SEQ ID NO: 30 - protein, 13 aa, Artificial
  VEGF-binding peptide
CXIQIMWDWECFR

SEQ ID NO: 31 - protein, 19 aa, Artificial
  VEGF-binding peptide v114
VEPNCDIHVMWEWECFERL

SEQ ID NO: 32 - protein, 41 aa, Artificial
  bidentate VEGF-binding peptide
SSKCGRWGETPCLAEGGGSGGGNFGYGKWEWDYGKWLEKVG

SEQ ID NO: 33 - protein, 41 aa, Artificial
  bidentate VEGF-binding peptide
VTSRYEMREDGSWKKGGGSGGGNFGYGKWEWDYGKWLEKVG

SEQ ID NO: 34 - protein, 38 aa, Artificial
  bidentate VEGF-binding peptide
AKRSSYSMWGAMPGGGSGGGNFGYGKWEWDYGKWLEKV

SEQ ID NO: 35 - protein, 40 aa, Artificial
  bidentate VEGF-binding peptide
SYVLKEWSVPSWGKPGGGSGGGNFGYGKWEWDYGKWLEKV

SEQ ID NO: 36 - protein, 35 aa, Artificial
  bidentate VEGF-binding peptide
SKLKTLLWQQGGGSGGGNFGYGKWEWDYGKWLEKV

SEQ ID NO: 37 - protein, 40 aa, Artificial
  bidentate VEGF-binding peptide
LLQKVMAKAGRGLPRGGGSGGGCPVQTMWDWECMRAFIEG

SEQ ID NO: 38 - protein, 30 aa, Artificial
  bidentate VEGF-binding peptide
GQSLAGGGSGGGCPVQTMWDWECMRAFIEG

SEQ ID NO: 39 - protein, 29 aa, Artificial
  bidentate VEGF-binding peptide
GQSLGGGSGGGCPVQTMWDWECMRAFIEG

SEQ ID NO: 40 - protein, 40 aa, Artificial
  bidentate VEGF-binding peptide
ALSKATEGVLRSLRGGGGSGGGCPVQTMWDWECMRAFIEG

SEQ ID NO: 41 - protein, 40 aa, Artificial
  bidentate VEGF-binding peptide
LLRVLTAAVGSSRGVGGGSGGGCPVQTMWDWECMRAFIEG

SEQ ID NO: 42 - protein, 15 aa, Artificial
  VEGF-binding peptide
SSKCGRWGETPCLAE

SEQ ID NO: 43 - protein, 15 aa, Artificial
  VEGF-binding peptide
VTSRYEMREDGSWKK

SEQ ID NO: 44 - protein, 13 aa, Artificial
  VEGF-binding peptide
AKRSSYSMWGAMP

SEQ ID NO: 45 - protein, 15 aa, Artificial
  VEGF-binding peptide
SYVLKEWSVPSWGKP

SEQ ID NO: 46 - protein, 10 aa, Artificial
  VEGF-binding peptide
SKLKTLLWQQ

SEQ ID NO: 47 - protein, 15 aa, Artificial
  VEGF-binding peptide
LLQKVMAKAGRGLPR

SEQ ID NO: 48 - protein, 5 aa, Artificial
  VEGF-binding peptide
GQSLA

SEQ ID NO: 49 - protein, 4 aa, Artificial
  VEGF-binding peptide
GQSL

SEQ ID NO: 50 - protein, 15 aa, Artificial
  VEGF-binding peptide
ALSKATEGVLRSLRG

SEQ ID NO: 51 - protein, 15 aa, Artificial
  VEGF-binding peptide
LLRVLTAAVGSSRGV

SEQ ID NO: 52 - protein, 7 aa, Artificial
  linker
GGGSGGG